SEQUENCE LISTING (32 sequences)

SEQ ID NO: 1 - DNA, 54 bp, Artificial Sequence
  /note=“Description of artificial sequence tal-finger nuclease target sequence within Rosa26”
tcgtgatctgcaactccagtctttctagaagatgggcgggagtcttctgggcag

SEQ ID NO: 2 - DNA, 7935 bp, Artificial Sequence
  /note=“Description of artificial sequence Sequence of Plasmid pCAG-venus-TalRosa1-Fok-EL”
gggtaccgggccccccctcgaggtcgacggtatcgataagcttgatatcgaattcgagctcggtacccgggggcgcgccggatctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgcccaagaagaagaggaaggtgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagggcggaggcggaggcggaggcacgcgtctggacaccggccagctgctgaagatcgccaagaggggcggcgtgaccgccgtggaggccgtgcacgcctggaggaacgccctgaccggcgcccctctgaacctgaccggtcagcaggtggtggccatcgccagccacgacggcggcaagcaggccctggagaccgtgcagaggctgctgcctgtgctgtgccaggcccacggcctgaccggtcagcaggtggtggccatcgccagccacgacggcggcaagcaggccctggagaccgtgcagaggctgctgcctgtgctgtgccaggcccacggcctgaccggtcagcaggtggtggccatcgccagccacgacggcggcaagcaggccctggagaccgtgcagaggctgctgcctgtgctgtgccaggcccacggcctgaccggtcagcaggtggtggccatcgccagcaacaacggcggcaagcaggccctggagaccgtgcagaggctgctgcctgtgctgtgccaggcccacggcctgaccggtcagcaggtggtggccatcgccagccacgacggcggcaagcaggccctggagaccgtgcagaggctgctgcctgtgctgtgccaggcccacggcctgaccggtcagcaggtggtggccatcgccagccacgacggcggcaagcaggccctggagaccgtgcagaggctgctgcctgtgctgtgccaggcccacggcctgaccggtcagcaggtggtggccatcgccagccacgacggcggcaagcaggccctggagaccgtgcagaggctgctgcctgtgctgtgccaggcccacggcctgaccggtgagcaggtggtggccatcgccagcaacatcggcggcaagcaggccctggagaccgtgcagaggctgctgcctgtgctgtgccaggcccacggcctgaccggtcagcaggtggtggccatcgccagcaacggcggcggcaagcaggccctggagaccgtgcagaggctgctgcctgtgctgtgccaggcccacggcctgaccggtcagcaggtggtggccatcgccagccacgacggcggcaagcaggccctggagaccgtgcagaggctgctgcctgtgctgtgccaggcccacggcctgaccggtcagcaggtggtggccatcgccagcaacggcggcggcaagcaggccctggagaccgtgcagaggctgctgcctgtgctgtgccaggcccacggcctgaccggtcagcaggtggtggccatcgccagcaacggcggcggcaagcaggccctggagaccgtgcagaggctgctgcctgtgctgtgccaggcccacggcctgaccggtcagcaggtggtggccatcgccagccacgacggcggcaagcaggccctggagaccgtgcagaggctgctgcctgtgctgtgccaggcccacggcctgaccggtcagcaggtggtggccatcgccagcaacggcggcggcaggcctgccctggagagcatcgtggcccagctgagcaggcctgaccctgccctggccggatccggcggcggcggcggcggcggccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatggagcgatatgtcgaagaaaatcaaacacgaaacaaacatctcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtgagacggaaatttaataacggcgagataaactttgctagcggatccacgcgtaaatgattgcagatccactagttctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatccactagttctagcctcgaggctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagct

SEQ ID NO: 3 - protein, 978 aa, Artificial Sequence
  /note=“Description of artificial sequence Aminoacid sequence of protein venus-TalRosa1-Fok-EL”
MPKKKRKVMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALSKDPNEKRDHMVLLEFVTAAGITLGMDELYKGGGGGGGTRLDTGQLLKIAKRGGVTAVEAVHAWRNALTGAPLNLTGQQVVAIASHDGGKQALETVQRLLPVLCQAHGLTGQQVVAIASHDGGKQALETVQRLLPVLCQAHGLTGQQVVAIASHDGGKQALETVQRLLPVLCQAHGLTGQQVVAIASNNGGKQALETVQRLLPVLCQAHGLTGQQVVAIASHDGGKQALETVQRLLPVLCQAHGLTGQQVVAIASHDGGKQALETVQRLLPVLCQAHGLTGQQVVAIASHDGGKQALETVQRLLPVLCQAHGLTGEQVVAIASNIGGKQALETVQRLLPVLCQAHGLTGQQVVAIASNGGGKQALETVQRLLPVLCQAHGLTGQQVVAIASHDGGKQALETVQRLLPVLCQAHGLTGQQVVAIASNGGGKQALETVQRLLPVLCQAHGLTGQQVVAIASNGGGKQALETVQRLLPVLCQAHGLTGQQVVAIASHDGGKQALETVQRLLPVLCQAHGLTGQQVVAIASNGGGRPALESIVAQLSRPDPALAGSGGGGGGGQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINFASGSTRK

SEQ ID NO: 4 - DNA, 8037 bp, Artificial Sequence
  /note=“Description of artificial sequence Sequence of Plasmid pCAG-venus-TalRosa2-Fok-KK”
gggtaccgggccccccctcgaggtcgacggtatcgataagcttgatatcgaattcgagctcggtacccgggggcgcgccggatctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgcccaagaagaagaggaaggtgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagggcggaggcggaggcggaggcacgcgtctggacaccggccagctgctgaagatcgccaagaggggcggcgtgaccgccgtggaggccgtgcacgcctggaggaacgccctgaccggcgcccctctgaacctgaccggtcagcaggtggtggccatcgccagccacgacggcggcaagcaggccctggagaccgtgcagaggctgctgcctgtgctgtgccaggcccacggcctgaccggtcagcaggtggtggccatcgccagcaacggcggcggcaagcaggccctggagaccgtgcagaggctgctgcctgtgctgtgccaggcccacggcctgaccggtcagcaggtggtggccatcgccagcaacaacggcggcaagcaggccctggagaccgtgcagaggctgctgcctgtgctgtgccaggcccacggcctgaccggtcagcaggtggtggccatcgccagccacgacggcggcaagcaggccctggagaccgtgcagaggctgctgcctgtgctgtgccaggcccacggcctgaccggtgagcaggtggtggccatcgccagcaacatcggcggcaagcaggccctggagaccgtgcagaggctgctgcctgtgctgtgccaggcccacggcctgaccggtgagcaggtggtggccatcgccagcaacatcggcggcaagcaggccctggagaccgtgcagaggctgctgcctgtgctgtgccaggcccacggcctgaccggtcagcaggtggtggccatcgccagccacgacggcggcaagcaggccctggagaccgtgcagaggctgctgcctgtgctgtgccaggcccacggcctgaccggtcagcaggtggtggccatcgccagcaacggcggcggcaagcaggccctggagaccgtgcagaggctgctgcctgtgctgtgccaggcccacggcctgaccggtcagcaggtggtggccatcgccagccacgacggcggcaagcaggccctggagaccgtgcagaggctgctgcctgtgctgtgccaggcccacggcctgaccggtcagcaggtggtggccatcgccagccacgacggcggcaagcaggccctggagaccgtgcagaggctgctgcctgtgctgtgccaggcccacggcctgaccggtgagcaggtggtggccatcgccagcaacatcggcggcaagcaggccctggagaccgtgcagaggctgctgcctgtgctgtgccaggcccacggcctgaccggtcagcaggtggtggccatcgccagcaacaacggcggcaagcaggccctggagaccgtgcagaggctgctgcctgtgctgtgccaggcccacggcctgaccggtcagcaggtggtggccatcgccagcaacggcggcggcaagcaggccctggagaccgtgcagaggctgctgcctgtgctgtgccaggcccacggcctgaccggtcagcaggtggtggccatcgccagccacgacggcggcaagcaggccctggagaccgtgcagaggctgctgcctgtgctgtgccaggcccacggcctgaccggtcagcaggtggtggccatcgccagcaacggcggcggcaggcctgccctggagagcatcgtggcccagctgagcaggcctgaccctgccctggccggatccggcggcggcggcggcggcggccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcaaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcataagactaattgtaatggagctgttcttagtgtagaagagcttttaattggtggagaaatgattaaagccggcacattaaccttagaggaagtgagacggaaatttaataacggcgagataaactttgctagcggatccacgcgtaaatgattgcagatccactagttctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatccactagttctagcctcgaggctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagct

SEQ ID NO: 5 - protein, 1012 aa, Artificial Sequence
  /note=“Description of artificial sequence Aminoacid sequence of protein venus-TalRosa2-Fok-KK”
MPKKKRKVMVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALSKDPNEKRDHMVLLEFVTAAGITLGMDELYKGGGGGGGTRLDTGQLLKIAKRGGVTAVEAVHAWRNALTGAPLNLTGQQVVAIASHDGGKQALETVQRLLPVLCQAHGLTGQQVVAIASNGGGKQALETVQRLLPVLCQAHGLTGQQVVAIASNNGGKQALETVQRLLPVLCQAHGLTGQQVVAIASHDGGKQALETVQRLLPVLCQAHGLTGEQVVAIASNIGGKQALETVQRLLPVLCQAHGLTGEQVVAIASNIGGKQALETVQRLLPVLCQAHGLTGQQVVAIASHDGGKQALETVQRLLPVLCQAHGLTGQQVVAIASNGGGKQALETVQRLLPVLCQAHGLTGQQVVAIASHDGGKQALETVQRLLPVLCQAHGLTGQQVVAIASHDGGKQALETVQRLLPVLCQAHGLTGEQVVAIASNIGGKQALETVQRLLPVLCQAHGLTGQQVVAIASNNGGKQALETVQRLLPVLCQAHGLTGQQVVAIASNGGGKQALETVQRLLPVLCQAHGLTGQQVVAIASHDGGKQALETVQRLLPVLCQAHGLTGQQVVAIASNGGGRPALESIVAQLSRPDPALAGSGGGGGGGQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINFASGSTRK

SEQ ID NO: 6 - DNA, 12565 bp, Artificial Sequence
  /note=“Description of artificial sequence gene targeting vector pRosa26.8-2”
caccgcattaccctgttatccctagcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctaggcgcgccctcgaggtgacctgcacgtctagggcgcagtagtccagggtttccttgatgatgtcatacttatcctgtcccttttttttccacagctcgcggttgaggacaaactcttcgcggtctttccagtactaggggatcgaaagagcctgctaaagcaaaaaagaagtcaccatgtcgtttactttgaccaacaagaacgtgattttcgttgccggtctgggaggcattggtctggacaccagcaaggagctgctcaagcgcgatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattacagctgagcgccggtcgctaccattaccagttggtctggtgtcaaaaataataataaccgggcaggccatgtctgcccgtatttcgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcggcctcgactctaggcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgagggctgcagatctgtagggcgcagtagtccagggtttccttgatgatgtcatacttatcctgtcccttttttttccacagctcgcggttgaggacaaactcttcgcggtctttccagtggggatcgacggtatcgataagctggccgctctagtggccgtacgggcccacctgccgggccacttaattaaatttaaatcacgtgctagcgcttaagcttgaagttcctattccgaagttcctattctctagaaagtataggaacttcggcgcgccgtcgacgtttaaacatgcatgaagttcctattccgaagttcctattctctagaaagtataggaacttcataaaacctgcaggcatgcaagcgatcgcggccggccaaggcccgcggggccactagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcaggtggtgttccacatttgggcctattctcagtccagggagttttacaacaatagatgtattgagaatccaacctaaagcttaactttccactcccatgaatgcctctctcctttttctccatttataaactgagctattaaccattaatggtttccaggtggatgtctcctcccccaatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagcttatcgatccgtcgacggtatcgataagcttgatatcgaattccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagct

SEQ ID NO: 7 - DNA, 3049 bp, Artificial Sequence
  /note=“Description of artificial sequence Sequence of plasmid pbs-Rosa-targetseq ”
gctggaaacatgcatgaagttcctattccgaagttcctattctctagaaagtataggaacttcataaaacctgcaggcatgcaagcgatcgcggccggccaaggcccgcggggccactagttctagagcggcctgatctgcaactccagtctttctagaagatgggcgggagtcttcgggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaa

SEQ ID NO: 8 - DNA, 6453 bp, Artificial sequence
  /note=“Description of artificial sequence pCAG-Tal-IX-Fok”
ggcgcgccggattcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaggtgcggtccaccgtcgctcagcaccatgaagcactggtggggcacggtttcacacacgcccatattgtggctctgtctcagcatcccgctgcactcgggactgtggccgtcaaatatcaggacatgatcgccgctctgcctgaggcaacccacgaagccattgtgggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatcttatgagacgacgtctcacggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaaccagctcgtgaaaagcgaactcgaagaaaagaaaagtgaactgcggcacaaactgaaatacgtcccacatgaatacattgagctgatcgagattgctaggaactccacccaggacagaatcctcgagatgaaagtgatggaattctttatgaaagtctacgggtatcggggcaagcacctgggcggatctcgcaaaccagatggggcaatctacactgtgggtagtcccatcgactatggcgtgattgtcgataccaaggcctacagtgggggttataatctgcccattggacaggctgacgagatgcagcgatacgtggaggaaaaccagacaagaaataagcatatcaaccccaatgagtggtggaaagtgtatcctagctccgtcactgaattcaagtttctcttcgtgtcaggccactttaagggaaactacaaagcacagctgaccaggctcaatcatattacaaactgcaatggcgccgtgctgagcgtcgaggaactgctcatcggcggagagatgatcaaggccggcacactcaccctggaggaggtccgccgaaaattcaataacggggaaatcaacttctgaacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatga

SEQ ID NO: 9 - protein, 176 aa, Artificial sequence
  /note=“Description of artificial sequence Nterm”
MGPKKKRKVAAADYKDDDDKPGGGGSGGGGVPASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQSGLDTGQLLKIAKRGGVTAVEAVHAWRNALTGAPLN

SEQ ID NO: 10 - protein, 78 aa, Artificial sequence
  /note=“Description of artificial sequence Cterm”
LTPQQVVAIASNGGGRPALESIVAQLSRPDPALARSALTNDHLVALACLGGRPALDAVKKGLPHAPALIKRTNRRIPE

SEQ ID NO: 11 - protein, 202 aa, Artificial sequence
  /note=“Description of artificial sequence Fok”
GSDRLNQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 12 - DNA, 7654 bp, Artificial sequence
  /note=“Description of artificial sequence pCAG-ArtTal1-Fok”
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaggtgcggtccaccgtcgctcagcaccatgaagcactggtggggcacggtttcacacacgcccatattgtggctctgtctcagcatcccgctgcactcgggactgtggccgtcaaatatcaggacatgatcgccgctctgcctgaggcaacccacgaagccattgtgggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatcttactccagaacaggtcgtcgcaatcgcaagtaacatcggcggaaaacaggccctcgaaaccgtccagagactcctccccgtgctgtgccaggcccacggactgaccccacagcaggtggtcgccatcgctagcaacggcggagggaagcaggctctggagaccgtgcagaggctgctccccgtcctgtgccaggcacatgggctcacacctcagcaggtggtcgcaattgcctccaatggtggcggaaaacaggccctggaaactgtgcagagactgctccccgtgctgtgccaggctcacggtctcacaccccagcaggtggtcgctatcgcatctcatgacgggggcaagcaggcactggagacagtgcagcggctgctccctgtcctgtgccaggcccacggactcactcctcagcaggtcgtcgccattgctagtaacggcggagggaaacaggctctggaaaccgtgcagcgcctgctccccgtgctgtgccaagcccacggcctgaccccccagcaggtggtcgcaatcgcctcaaacaatggtggcaagcaggccctggagactgtgcagcgactgctcccagtgctgtgccaggcccatggactcacaccacagcaggtcgtcgctattgcaagcaacaatggagggaaacaggcactggaaacagtccagaggctgctccccgtgctgtgccaagcgcatggactcactccccagcaggtcgtcgccatcgcttccaataacggcggcaagcaggccctggagaccgtccagagactgctccccgtgctgtgccaagctcacggactcacacctgagcaggtcgtggcaatcgcctctaacattggagggaaacaggccctggaaactgtacagcggctgctccccgtgctgtgccaagcacacggactcactccacagcaggtcgtggccattgcaagtcatgacggaggcaagcaggccctggaaacagtgcagcgcctgctccctgtgctgtgccaggctcatggtctgactcctcagcaggtggtggccatcgcttccaacaatggagggaagcaggccctggagaccgtacagagactgctccccgtgctgtgccaagcgcacggtctgacccctcagcaggtcgtcgcaatcgccagcaatggcgggggcaagcaggctctcgaaaccgtccagcggctcctcccagtcctctgtcaggctcacggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaaccagctcgtgaaaagcgaactcgaagaaaagaaaagtgaactgcggcacaaactgaaatacgtcccacatgaatacattgagctgatcgagattgctaggaactccacccaggacagaatcctcgagatgaaagtgatggaattctttatgaaagtctacgggtatcggggcaagcacctgggcggatctcgcaaaccagatggggcaatctacactgtgggtagtcccatcgactatggcgtgattgtcgataccaaggcctacagtgggggttataatctgcccattggacaggctgacgagatgcagcgatacgtggaggaaaaccagacaagaaataagcatatcaaccccaatgagtggtggaaagtgtatcctagctccgtcactgaattcaagtttctcttcgtgtcaggccactttaagggaaactacaaagcacagctgaccaggctcaatcatattacaaactgcaatggcgccgtgctgagcgtcgaggaactgctcatcggcggagagatgatcaaggccggcacactcaccctggaggaggtccgccgaaaattcaataacggggaaatcaacttctgaacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgaggcgcgccggattc

SEQ ID NO: 13 - DNA, 8164 bp, Artificial sequence
  /note=“Description of artificial sequence pCAG-AvrBs-Fok”
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaggtgcggtccaccgtcgctcagcaccatgaagcactggtggggcacggtttcacacacgcccatattgtggctctgtctcagcatcccgctgcactcgggactgtggccgtcaaatatcaggacatgatcgccgctctgcctgaggcaacccacgaagccattgtgggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatcttactcccgaacaggtcgtggctatcgcttcccatgatggtggtaaacaggccctcgaaaccgtccagagactgctgcccgtgctctgccaggcacacggactgacccctcagcaggtggtcgccatcgctagcaacggcggagggaagcaggctctggagaccgtgcagcggctgctccccgtcctgtgccaggcacatggtctcacacctcagcaggtggtcgcaattgccagcaattccggtggcaaacaggccctggagactgtgcagcgcctgctccccgtgctgtgccaggctcacggactcacccccgagcaggtggtcgctatcgcatccaacggagggggcaagcaggcactggaaacagtgcagcgactgctccctgtcctgtgccaggcccatggactcactccagagcaggtcgtggccatcgcttctaatattggcggaaaacaggcactggaaaccgtgcaggccctgctgcccgtgctgtgccaggcacacggactcacacctgagcaggtggtcgcaatcgccagtaacattgggggcaagcaggctctggaaactgtgcaggcactgctcccagtcctgtgccaggctcacggcctgacccccgagcaggtcgtcgctatcgcatcaaacatcggcggaaaacaggccctggaaacagtgcaggctctgttacccgtgctgtgccaggcccacggcctgactccagagcaggtggtcgccattgctagccatgacggtggcaagcaggctctggaaaccgtacagaggctgctccccgtgctgtgccaagcccatggcctgacacctgagcaggtcgtggcaatcgcctcccatgatggtggaaaacaggccctggaaactgtgcagagactgctccccgtgctgtgccaagcgcacggactcaccccacagcaggtggtcgctattgcatctaacgggggtggcaagcaggcactggagacagtgcagcggctgctccctgtgctgtgccaggcacatggcctgactccagagcaagtggtcgccatcgcttctaatagtggagggaaacaggcactggaaaccgtacaggccctgttacccgtgctgtgccaagctcatggcctcacacctgagcaggtcgtcgcaattgcctcaaacagcggtggcaagcaggccctggaaactgtccagcgcctgctcccagtgctgtgccaagcgcatggcctcacccccgagcaggtcgtggctatcgcaagtcatgacggagggaaacaggccctggaaacagtacagcgactgctccccgtgctgtgccaagcacacggactgactccagagcaggtcgtcgccattgcttcacatgatggcggcaagcaggccctggaaaccgtccagcggctgctccccgtgctgtgccaagcgcacggcttaacacctgagcaagtcgtggcaatcgccagtcatgacggagggaagcaggccctggaaactgttcagaggctgctccccgtgctgtgccaagcgcacggtctgacaccccagcaggtcgtggcaattgcctccaatggtggaggaaggcctgccctggagaccgtgcagagactgctcccagtgctgtgccaggctcatggactgacacccgagcaggtcgtcgcaatcgcttctcatgatggcggcaagcaggctctggaaaccgtgcagcgactcctccccgtcctctgtcaggctcacggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaaccagctcgtgaaaagcgaactcgaagaaaagaaaagtgaactgcggcacaaactgaaatacgtcccacatgaatacattgagctgatcgagattgctaggaactccacccaggacagaatcctcgagatgaaagtgatggaattctttatgaaagtctacgggtatcggggcaagcacctgggcggatctcgcaaaccagatggggcaatctacactgtgggtagtcccatcgactatggcgtgattgtcgataccaaggcctacagtgggggttataatctgcccattggacaggctgacgagatgcagcgatacgtggaggaaaaccagacaagaaataagcatatcaaccccaatgagtggtggaaagtgtatcctagctccgtcactgaattcaagtttctcttcgtgtcaggccactttaagggaaactacaaagcacagctgaccaggctcaatcatattacaaactgcaatggcgccgtgctgagcgtcgaggaactgctcatcggcggagagatgatcaaggccggcacactcaccctggaggaggtccgccgaaaattcaataacggggaaatcaacttctgaacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgaggcgcgccggattc

SEQ ID NO: 14 - DNA, 7756 bp, Artificial sequence
  /note=“Description of artificial sequence pCAG-TalRab1-Fok”
ggcgcgccggattcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaggtgcggtccaccgtcgctcagcaccatgaagcactggtggggcacggtttcacacacgcccatattgtggctctgtctcagcatcccgctgcactcgggactgtggccgtcaaatatcaggacatgatcgccgctctgcctgaggcaacccacgaagccattgtgggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatctgacaccccagcaggtggtggccattgctagcaacaatgggggcaagcaggctctggagacagtgcagcgcctgctgcctgtgctgtgccaggctcacggactgactccacagcaggtggtggccatcgcttccaacggagggggcaaacaggctctggaaacagtgcagaggctgctgcccgtgctgtgccaggctcatggactgacacctcagcaggtcgtcgccattgcttctaacaatggagggaagcaggctctggagactgtgcagagactgctgccagtgctgtgccaggcccatggactgacccctcagcaggtcgtggctatcgctagtcatgatggcggaaaacaggctctggaaactgtgcagcggctgctccccgtgctgtgccaggcccacggactgactccagaacaggtcgtggccatcgctagcaacatcgggggcaagcaggctctggaaacagtccagcgcctgttacccgtgctgtgccaggcacacggcctcacacctcagcaggtcgtggcaattgcttcccatgacggagggaaacaggctctggagaccgtccagaggctgctccccgtgctgtgccaagctcacggcctcacccctcagcaggtggtcgctatcgcttctcatgatggcggaaagcaggctctggaaaccgtgcagagactgctccctgtgctgtgccaagcccacggcctcactccagaacaggtggtcgccatcgctagtaacattgggggcaaacaggctctggaaacagtacagcggctgttacccgtgctgtgccaagcccatggactgacacctgaacaggtggtggctatcgctagcaatatcggagggaagcaggctctggaaactgtccagcgcctgctcccagtgctgtgccaggcacatggactgacccctgaacaggtggtggcaatcgcttccaacattggcggaaaacaggccctggaaaccgtccagaggctgttacccgtgctgtgccaagcgcatggactgactccagagcaggtcgtcgccatcgcttctaatattgggggcaagcaggccctggaaacagtccagagactgttgcccgtgctgtgccaagcccacggtctcacacctcagcaggtggtcgcaatcgctagtcatgacggagggaagcaggccctggagacagtgcagcggctgcttcccgtgctgtgccaagcacatggcctcacaccccagcaggtcgtggcaatcgcctccaatggcggagggaagcaggccctggagacggtgcagagactgttacctgtgctgtgccaggcccatggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaaccagctcgtgaaaagcgaactcgaagaaaagaaaagtgaactgcggcacaaactgaaatacgtcccacatgaatacattgagctgatcgagattgctaggaactccacccaggacagaatcctcgagatgaaagtgatggaattctttatgaaagtctacgggtatcggggcaagcacctgggcggatctcgcaaaccagatggggcaatctacactgtgggtagtcccatcgactatggcgtgattgtcgataccaaggcctacagtgggggttataatctgcccattggacaggctgacgagatgcagcgatacgtggaggaaaaccagacaagaaataagcatatcaaccccaatgagtggtggaaagtgtatcctagctccgtcactgaattcaagtttctcttcgtgtcaggccactttaagggaaactacaaagcacagctgaccaggctcaatcatattacaaactgcaatggcgccgtgctgagcgtcgaggaactgctcatcggcggagagatgatcaaggccggcacactcaccctggaggaggtccgccgaaaattcaataacggggaaatcaacttctgaacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatga

SEQ ID NO: 15 - DNA, 7858 bp, Artificial sequence
  /note=“Description of artificial sequence pCAG-TalRab2-Fok”
ggcgcgccggattcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagatccttaattaataatacgactcactataggggccgccaccatgggacctaagaaaaagaggaaggtggcggccgctgactacaaggatgacgacgataaaccaggtggcggaggtagtggcggaggtggggtacccgccagtccagcagcccaggtggatctgagaaccctcggctacagccagcagcagcaggagaagatcaaaccaaaggtgcggtccaccgtcgctcagcaccatgaagcactggtggggcacggtttcacacacgcccatattgtggctctgtctcagcatcccgctgcactcgggactgtggccgtcaaatatcaggacatgatcgccgctctgcctgaggcaacccacgaagccattgtgggcgtcggaaagcagtggagcggtgccagagcactcgaagcactcctcaccgtcgccggggaactgcggggtccaccactccagtccggactggacactggacagctgctgaagatcgctaaacgcggcggagtgacagctgtggaagctgtgcacgcttggaggaatgctctgacaggagccccactgaatctgacaccccagcaggtggtggccattgctagcaacaatgggggcaagcaggctctggagacagtgcagcgcctgctgcctgtgctgtgccaggctcacggactgactccacagcaggtggtggccatcgcttccaacaatggagggaaacaggctctggaaacagtgcagaggctgctgcccgtgctgtgccaggctcatggactgacacctcagcaggtcgtcgccattgcttctaacggcggagggaagcaggctctggagactgtgcagagactgctgccagtgctgtgccaggcccatggactgacccctcagcaggtcgtggctatcgctagtaacaatggcggaaaacaggctctggaaactgtgcagcggctgctccccgtgctgtgccaggcccacggcctcactccacagcaggtcgtcgctatcgcctctaataacgggggcaagcaggctctggagacagtacagcgcctgttacccgtgctgtgccaggcacacggcctcacacctcagcaggtcgtggcaatcgcttcccatgacggagggaaacaggctctggaaacggtccagaggctgctccccgtgctgtgccaagctcacggcctcacccctcagcaggtggtcgctattgcttctcatgatggcggaaagcaggctctggagaccgtgcagagactgctccctgtgctgtgccaagcccacggcctgactccacagcaggtcgtggccatcgctagtcatgacgggggcaaacaggctctggaaacagtacagcggctgttacccgtgctgtgccaagcccatggcctcacacctcagcaagtcgtcgctatcgctagcaacaatggagggaagcaggctctggagacggtgcagcgcctgctcccagtgctgtgccaagctcatggcctcacccctcagcaagtcgtcgcaattgcttccaataacggcggaaaacaggctctggaaaccgtccagaggctgctgcccgtgctgtgccaagcacatggcttaactccacagcaagtggtggccattgcttctaatgggggcggaaagcaggccctggagacagtccagagactgttgcccgtgctgtgccaagcgcatggactgacacctgaacaggtcgtcgctatcgctagtaatattgggggcaaacaggccctggaaacagtgcagcggctgcttcccgtgctgtgccaggcgcatggactcacaccccagcaggtcgtcgcaatcgcctctaataacggagggaagcaggccctggaaaccgtgcagagactgttacctgtgctgtgccaggcacatggtctgacaccacagcaggtggtcgcaattgctagcaatggcggagggaagcaggccctggagactgtccagagactgctacccgtgctgtgccaagcgcacggcctgaccccacagcaggtcgtcgctattgcttctaatggcggagggcggcctgctctggagagcattgtggctcagctgtccaggcccgatcctgccctggctagatccgcactcactaacgatcatctggtcgctctcgcttgcctcggtggacggcccgctctggacgcagtcaaaaagggtctcccccatgctcccgcactgatcaagagaaccaacaggagaattcctgagggatccgatcgtttaaaccagctcgtgaaaagcgaactcgaagaaaagaaaagtgaactgcggcacaaactgaaatacgtcccacatgaatacattgagctgatcgagattgctaggaactccacccaggacagaatcctcgagatgaaagtgatggaattctttatgaaagtctacgggtatcggggcaagcacctgggcggatctcgcaaaccagatggggcaatctacactgtgggtagtcccatcgactatggcgtgattgtcgataccaaggcctacagtgggggttataatctgcccattggacaggctgacgagatgcagcgatacgtggaggaaaaccagacaagaaataagcatatcaaccccaatgagtggtggaaagtgtatcctagctccgtcactgaattcaagtttctcttcgtgtcaggccactttaagggaaactacaaagcacagctgaccaggctcaatcatattacaaactgcaatggcgccgtgctgagcgtcgaggaactgctcatcggcggagagatgatcaaggccggcacactcaccctggaggaggtccgccgaaaattcaataacggggaaatcaacttctgaacgcgtaaatgattgcagatccactagttctagaattccagctgagcgccggtcgctaccattaccagttggtctggtgtcaaaaataataataaccgggcaggggggatctgcatggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgccagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctgcggccgccactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatga

SEQ ID NO: 16 - protein, 864 aa, Artificial sequence
  /note=“Description of artificial sequence ArtTal1-Fok”
MGPKKKRKVAAADYKDDDDKPGGGGSGGGGVPASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQSGLDTGQLLKIAKRGGVTAVEAVHAWRNALTGAPLNLTPEQVVAIASNIGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQRLLPVLCQAHGLTPQQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALARSALTNDHLVALACLGGRPALDAVKKGLPHAPALIKRTNRRIPEGSDRLNQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 17 - protein, 1034 aa, Artificial sequence
  /note=“Description of artificial sequence AvrBs-Fok”
MGPKKKRKVAAADYKDDDDKPGGGGSGGGGVPASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQSGLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPQQVVAIASNSGGKQALETVQRLLPVLCQAHGLTPEQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNSGGKQALETVQALLPVLCQAHGLTPEQVVAIASNSGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGRPALETVQRLLPVLCQAHGLTPEQVVAIASHDGGKQALETVQRLLPVLCQAHGLTPQQVVAIASNGGGRPALESIVAQLSRPDPALARSALTNDHLVALACLGGRPALDAVKKGLPHAPALIKRTNRRIPEGSDRLNQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 18 - protein, 898 aa, Artificial sequence
  /note=“Description of artificial sequence TalRab1-Fok”
MGPKKKRKVAAADYKDDDDKPGGGGSGGGGVPASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQSGLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASHDGGKQALETVQRLLPVLCQAHGLTPEQVVAIASNIGGKQALETVQRLLPVLCQAHGLTPQQVVAIASHDGGKQALETVQRLLPVLCQAHGLTPQQVVAIASHDGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPEQVVAIASNIGGKQALETVQRLLPVLCQAHGLTPQQVVAIASHDGGKQALETVQRLLPVLCQAHGLTPQQVVAIASNGGGKQALETVQRLLPVLCQAHGLTPQQVVAIASNGGGRPALESIVAQLSRPDPALARSALTNDHLVALACLGGRPALDAVKKGLPHAPALIKRTNRRIPEGSDRLNQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 19 - protein, 932 aa, Artificial sequence
  /note=“Description of artificial sequence TalRab2-Fok”
MGPKKKRKVAAADYKDDDDKPGGGGSGGGGVPASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQSGLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASHDGGKQALETVQRLLPVLCQAHGLTPQQVVAIASHDGGKQALETVQRLLPVLCQAHGLTPQQVVAIASHDGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALARSALTNDHLVALACLGGRPALDAVKKGLPHAPALIKRTNRRIPEGSDRLNQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 20 - DNA, 7374 bp, Artificial sequence
  /note=“Description of artificial sequence pCMV-ArtTal1-Fok-Reporter”
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgacgacactgcagagacctacttcactaacaaccggtatggtcgcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctataaaaccggtacagttcggccaccatggtcgtattctgggacgttttcacactcttctaacgtcccagaatactcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattacagctgagcgccggtcgctaccattaccagttggtctggtgtcaaaaataataataaccgggcaggccatgtctgcccgtatttcgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcggcctcgactctaggcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagcttgcatgcctgcaggt

SEQ ID NO: 21 - DNA, 7384 bp, Artificial sequence
  /note=“Description of artificial sequence pCMV-AvrBs3-Fok-Reporter”
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgacgacactgcagagacctacttcactaacaaccggtatggtcgcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctataaaaccggtacagttcggccaccatggtcgtatataaacctaaccctcttttcacactcttctaagagggttaggtttatatacgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattacagctgagcgccggtcgctaccattaccagttggtctggtgtcaaaaataataataaccgggcaggccatgtctgcccgtatttcgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcggcctcgactctaggcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagcttgcatgcctgcaggt

SEQ ID NO: 22 - DNA, 7374 bp, Artificial sequence
  /note=“Description of artificial sequence pCMV-TalRab1-Fok-Reporter”
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgacgacactgcagagacctacttcactaacaaccggtatggtcgcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctataaaaccggtacagttcggccaccatggtcgtgtgcaccaaaacttttcacactcttctaagttttggtgcacacgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattacagctgagcgccggtcgctaccattaccagttggtctggtgtcaaaaataataataaccgggcaggccatgtctgcccgtatttcgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcggcctcgactctaggcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagcttgcatgcctgcaggt

SEQ ID NO: 23 - DNA, 7377 bp, Artificial sequence
  /note=“Description of artificial sequence pCMV-TalRab2-Fok-Reporter”
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgacgacactgcagagacctacttcactaacaaccggtatggtcgcgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctataaaaccggtacagttcggccaccatggtcgatggtggcccggtagttttcacactcttctcactaccgggccaccacgagtagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattacagctgagcgccggtcgctaccattaccagttggtctggtgtcaaaaataataataaccgggcaggccatgtctgcccgtatttcgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcggcctcgactctaggcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagcttgcatgcctgcaggt

SEQ ID NO: 24 - protein, 34 aa, Artificial sequence
  /note=“Description of artificial sequence Tal effector motif (repeat) #11 derived from the Xanthomonas Hax3 protein with amino acids N12 and I13”
LTPEQVVAIASNIGGKQALETVQRLLPVLCQAHG

SEQ ID NO: 25 - protein, 34 aa, Artificial sequence
  /note=“Description of artificial sequence Tal effector motif (repeat) #5 derived from the Hax3 protein with amino acids H12 and D13”
LTPQQVVAIASHDGGKQALETVQRLLPVLCQAHG

SEQ ID NO: 26 - protein, 34 aa, Artificial sequence
  /note=“Description of artificial sequence Tal effector motif (repeat) #4 from the Xanthomonas Hax4 protein with amino acids N12 and G13”
LTPQQVVAIASNGGGKQALETVQRLLPVLCQAHG

SEQ ID NO: 27 - protein, 34 aa, Artificial sequence
  /note=“Description of artificial sequence Tal effector motif (repeat) #4 from the Hax4 protein with replacement of the amino acids 12 into N and 13 into N”
LTPQQVVAIASNNGGKQALETVQRLLPVLCQAHG

SEQ ID NO: 28 - protein, 34 aa, Artificial sequence
  /note=“Description of artificial sequence invariable first Tal-repeat from the Hax3 protein”
LDTGQLLKIAKRGGVTAVEAVHAWRNALTGAPLN

SEQ ID NO: 29 - protein, 34 aa, Artificial sequence
  /note=“Description of artificial sequence last Tal-repeat from the Hax3 protein”
LTPEQVVAIASNGGGRPALESIVAQLSRPDPALA

SEQ ID NO: 30 - DNA, 9489 bp, Artificial sequence
  /note=“Description of artificial sequence Vector pROSA26.3-3”
caccgcattaccctgttatccctagcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctaggcgcgcccgggctgcagatctgtagggcgcagtagtccagggtttccttgatgatgtcatacttatcctgtcccttttttttccacagctcgcggttgaggacaaactcttcgcggtctttccagtggggatcgacggtatcgataagctggccgctctaggatccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaagaattcaaggcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcaggtggtgttccacatttgggcctattctcagtccagggagttttacaacaatagatgtattgagaatccaacctaaagcttaactttccactcccatgaatgcctctctcctttttctccatttataaactgagctattaaccattaatggtttccaggtggatgtctcctcccccaatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagcttatcgataccgtcgacggtatcgataagcttgatccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagct

SEQ ID NO: 31 - DNA, 460 bp, Artificial sequence
  /note=“Description of artificial sequence Rosa26 5′-probe”
gcccttcttctcagctacctttacacaccattgcaccgctcttgcccagagagaaaggctctccttcatctagtcgaccccactacctttttaatgtcttccctgggtcaggactcttcccctccccctactctggtctcccctttttgcctgggtattgcctactccacgtttatacccttttcaggagaggcctcccaaccctgctctcaaaatacacatacttttttttctgtccctgagccccccacctcccctgttcttgcggccttgtgacaactctggtcgctcgtgggggcccagtcctcccctccataatcttcctgaacgcctctcctctggttttccagttcctatctcagatggctgctgcttttcccacaccaaagacattaccttcgccacccccacctcacattcttggactccctgtggcgtatgccccagtatccttaagggc

SEQ ID NO: 32 - DNA, 730 bp, Artificial sequence
  /note=“Description of artificial sequence Venus probe”
gatccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaga

(19) United States
(12) Patent Application Publication
(10) Pub. No.: US 2013/0212725 A1
(43) Pub. Date: Aug. 15, 2013

(54) FUSION PROTEINS COMPRISING A DNA-BINDING DOMAIN OF A TAL EFFECTOR PROTEIN AND A NON-SPECIFIC CLEAVAGE DOMAIN OF A RESTRICTION NUCLEASE AND THEIR USE

(71) Applicants: Ralf Kühn, Freising (DE); Wolfgang Wurst, Munchen (DE); Melanie Meyer, Olching (DE)

(72) Inventors: Ralf Kühn, Freising (DE); Wolfgang Wurst, Munchen (DE); Melanie Meyer, Olching (DE)

(73) Assignee: HELMHOLTZ ZENTRUM MUNCHEN DEUTSCHES FORSCHUNGSZENTRUM FUR GESUNDHEIT UND, Neuherberg (DE)

(21) Appl. No.: 13/702,231

(22) PCT Filed: Jun. 7, 2011
(86) PCT No.: PCT/EP2011/059370

§ 371 Date: Apr. 18, 2013

Related U.S. Application Data
(60) Provisional application No. 61/352,103, filed on Jun. 7, 2010.

(30) Foreign Application Priority Data
Jun. 7, 2010 (EP) 10005863.5

(51) Int. Cl. C12N 15/62

(52) U.S. Cl. CPC C12N 15/62
USPC 800/21; 435/455; 435/463; 435/468; 435/471; 435/470; 435/196

(57) ABSTRACT

The present invention relates to a method of modifying a target sequence in the genome of a eukaryotic cell, the method comprising the step: (a) introducing into the cell a fusion protein comprising a DNA-binding domain of a Tal effector protein and a non-specific cleavage domain of a restriction nuclease or a nucleic acid molecule encoding the fusion protein in expressible form, wherein the fusion protein specifically binds within the target sequence and introduces a double strand break within the target sequence. The present invention further relates to the method of the invention, wherein the modification of the target sequence is by homologous recombination with a donor nucleic acid sequence further comprising the step: (b) introducing a nucleic acid molecule into the cell, wherein the nucleic acid molecule comprises the donor nucleic acid sequence and regions homologous to the target sequence. The present invention also relates to a method of producing a non-human mammal or vertebrate carrying a modified target sequence in its genome. Furthermore, the present invention relates to a fusion protein comprising a DNA-binding domain of a Tal effector protein and a non-specific cleavage domain of a restriction nuclease.

The present invention relates to a method of modifying a target sequence in the genome of a eukaryotic cell, the method comprising the step: (a) introducing into the cell a fusion protein comprising a DNA-binding domain of a Tal effector protein and a non-specific cleavage domain of a restriction nuclease or a nucleic acid molecule encoding the fusion protein in expressible form, wherein the fusion protein specifically binds within the target sequence and introduces a double strand break within the target sequence. The present invention further relates to the method of the invention, wherein the modification of the target sequence is by homologous recombination with a donor nucleic acid sequence further comprising the step: (b) introducing a nucleic acid molecule into the cell, wherein the nucleic acid molecule comprises the donor nucleic acid sequence and regions homologous to the target sequence. The present invention also relates to a method of producing a non-human mammal or vertebrate carrying a modified target sequence in its genome. Furthermore, the present invention relates to a fusion protein comprising a Tal effector protein and a non-specific cleavage domain of a restriction nuclease.

In this specification, a number of documents including patent applications and manufacturer's manuals is cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

With the complete elucidation of the human, mouse and other mammalian genome sequences a major challenge is the functional characterization of every gene within the genome and the identification of gene products and their molecular interaction network. In the past two decades the mouse has developed into the prime mammalian genetic model to study human biology and disease because methods are available that allow the production of targeted, predesigned mouse mutants. This reverse genetics approach that enables the production of germ line and conditional knockout mice by gene targeting, relies on the use of murine embryonic stem (ES) cell lines. ES cell lines exhibit unique properties such that they are able, once established from the inner cell mass of a mouse blastocyst, to renew indefinitely in cell culture while retaining their early pluripotent differentiation state. This property allows to grow ES cells in large numbers and, since most mutagenesis methods are inefficient, to select rare genetic variants that are expanded into a pure stem cell clone that harbours a specific genetic alteration in the target gene. Upon introduction of ES cells into mouse blastocysts and subsequent embryo transfer these cells contribute to all cell types of the developing chimaeric embryo, including the germ line. By mating of germ line chimaeras to normal mice a genetic modification engineered in ES cells is inherited to their offspring and thereby transferred into the mouse germ line.

The basis for reverse mouse genetics was initially established in the decade of 1980-90 in three steps and the basic scheme that is followed since that time is essentially unchanged. The first of these steps was the establishment of ES cell lines from cultured murine blastocysts and of culture conditions that maintain their pluripotent differentiation state in vitro (Evans M J, Kaufman M H., Nature 1981; 292:154-6; Martin G R. Proc Natl Acad Sci USA 1981; 78:7634-8). A few years later it was first reported that ES cells, upon microinjection into blastocysts, are able to colonize the germ line in chimaeric mice (Bradley A, Evans M, Kaufman M H, Robertson E., Nature 1984; 309:255-6; Gossler A, Doetschman T, Korn R, Serfling E, Kemler R., Proc Natl Acad Sci USA 1986; 83:9065-9). The third step concerns the technology to introduce pre-planned, inactivating mutations into target genes in ES cells by homologous recombination between a gene targeting vector and endogenous loci (gene targeting). Gene targeting allows the introduction of pre-designed, site-specific modifications into the mouse genome (Capecchi M R. Trends Genet 1989; 5:70-6). Since the first demonstration of homologous recombination in ES cells in 1987 (Thomas K R, Capecchi M R., Cell 1987; 51:503-12) and the establishment of the first knockout mouse strain in 1989 (Schwartzberg P L, Goff S P, Robertson E J., Science 1989; 246:799-803) gene targeting was adopted to many other genes and has been used in the last decades to generate more than 3000 knockout mouse strains that provided a wealth of information on in vivo gene functions (Collins F S, Rossant J, Wurst W., Cell 2007; 128:9-13; Capecchi, M. R., Nat Rev Genet 2005; 6: 507-12).

Targeted gene inactivation in ES cells can be achieved through the insertion of a selectable marker (mostly the neomycin phosphotransferase gene, neo) into an exon of the target gene or the replacement of one or more exons. The mutant allele is initially assembled in a specifically designed gene targeting vector such that the selectable marker is flanked at both sides with genomic segments of the target gene that serve as homology regions to initiate homologous recombination. The frequency of homologous recombination increases with the length of these homology arms. Usually arms with a combined length of 10-15 kb are cloned into standard, high copy plasmid vectors that accommodate up to 20 kb of foreign DNA. To select against random vector integrations a negative selectable marker, such as the Herpes simplex thymidine kinase or diphtheria toxin gene, can be included at one end of the targeting vector. Upon electroporation of such a vector into ES cells and the selection of stable integrants, clones that underwent a homologous recombination event can be identified through the analysis of genomic DNA using a PCR or Southern blot strategy. Using such standard gene targeting vectors the efficiency at which homologous recombinant ES cell clones are obtained is the range of 0.1% to 10% as compared to the number of stable transfected (Neo resistant) ES cell clones. This rate depends on the length of the vector homology region, the degree of sequence identity of this region with the genomic DNA of the ES cell line and likely on the differential accessibility of individual genomic loci to homologous recombination. Optimal rates are achieved with longer homology regions and by the use of genomic fragments that exhibit sequence identity to the genome of the ES cell line, i.e. both should be isogenic and derived from the same inbred mouse strain (te Riele H, Maandag E R, Berns A. 1992. Proc Natl Acad Sci USA 89:5128-5132). Since the frequency of stable transfection of ES cells by electroporation is about 10⁻⁴ (i.e. 1 Neo resistant cone from 10.000 electroporated cells), the absolute efficiency of obtaining homologous recombinant ES cells falls in the range of 10⁻⁵-10⁻⁷ (Cheah S S, Behringer R R., Methods Mol Biol 2000; 136: 455-63; DeChiara T M.; Methods Mol Biol 2001; 158: 19-45; Hasty P, Abuin A, Bradley A., 2000, In Gene Targeting: a practical approach, ed. A L Joyner, pp. 1-35. Oxford: Oxford University Press; Nagy A, Gertsenstein M, Vintersten K, Behringer R., 2003. Manipulating the Mouse Embryo. Cold Spring Harbour, N.Y.: Cold Spring Harbour Laboratory Press)

Upon the isolation of recombinant ES cell clones modified ES cells are injected into blastocysts to transmit the mutant allele through the germ line of chimaeras and to establish a mutant strain. Through interbreeding of heterozygous mutants homozygotes are obtained that can be used for phenotype analysis.

Using the “classical” gene targeting approach described above germ line mutants are obtained that harbour the knockout mutation in all cells throughout development. This strategy identifies the first essential function of a gene during ontogeny. If the gene product fulfils an important role in development its inactivation can lead to embryonic lethality precluding further analysis in adult mice. In general about 30% of all knockout mouse strains exhibit an embryonic lethal phenotype, for specific classes of genes, e.g. those regulating angiogenesis, this rate can reach 100%. To avoid embryonic lethality and to study gene function only in specific cell types Gu et al. (Gu H, Marth J D, Orban P C, Mossmann H, Rajewsky K., Science 1994; 265:103-6) introduced a modified, conditional gene targeting scheme that allows to restrict gene inactivation to specific cell types or developmental stages. In a conditional mutant, gene inactivation is achieved by the insertion of two 34 bp recognition (loxP) sites of the site-specific DNA recombinase Cre into introns of the target gene such that recombination results in the deletion of loxP-flanked exons. Conditional mutants initially require the generation of two mouse strains: one strain harbouring a loxP flanked gene segment obtained by gene targeting in ES cells and a second, transgenic strain expressing Cre recombinase in one or several cell types. The conditional mutant is generated by crossing these two strains such that target gene inactivation occurs in a spatial and temporal restricted manner, according to the pattern of recombinase expression in the Cre transgenic strain (Nagy A, Gertsenstein M, Vintersten K, Behringer R. 2003. Manipulating the Mouse Embryo, third edition ed. Cold Spring Harbour, N.Y.: Cold Spring Harbour Laboratory Press; Torres R M, Kuhn R. 1997. Laboratory protocols for conditional gene targeting. Oxford: Oxford University Press). Conditional mutants have been used to address various biological questions which could not be resolved with germ line mutants, often because a null allele results in an embryonic or neonatal lethal phenotype.

Taken together, gene targeting in ES cells has revolutionised the in vivo analysis of mammalian gene function using the mouse as genetic model system. However, since germ line competent ES cell lines that can be genetically modified could be established only from mice, this reverse genetics approach is presently restricted to this rodent species. The exception from this rule is achieved by homologous recombination in primary cells from pig and sheep followed by the transplantation of nuclei from recombined somatic cells into enucleated oocytes (cloning) (Lai L, Prather R S. 2003. Reprod Biol Endocrinol 2003; 1:82; Gong M, Rong Y S. 2003. Curr Opin Genet Dev 13:215-220). Since this methodology is inefficient and time consuming it does not have the potential to develop into a simple routine procedure.

Although the generation of targeted mouse mutants via genome engineering in ES cells and the derivation of germ line transmitting chimaeras is established as a routine procedure this approach typically requires 1-2 years of hands on work for vector construction, ES cell culture and selection and the breeding of chimaeras. Typical problems that are encountered during a gene targeting project are the low efficiency of homologous recombination in ES cells and the loss of the germ line competence of ES cells during the long in vitro culture and selection phase. Therefore, the successful generation of even a single line of knockout mice requires considerable time, the combined efforts of specialists in molecular biology, ES cell culture and embryo manipulation, and the associated technical infrastructure.

Experiments in model systems have demonstrated that the frequency of homologous recombination of a gene targeting vector is strongly increased if a double-strand break is induced within its chromosomal target sequence. Using the yeast homing endonuclease I-SceI, that cuts DNA at an 18 base pair-long recognition site, it was initially shown that homologous recombination and gene targeting are stimulated over 1000-fold in mammalian cells when a recognition site is inserted into a target gene and I-SceI is expressed in these cells (Rouet, P., Smih, F., Jasin, M.; Mol Cell Biol 1994; 14: 8096-8106; Rouet, P., Smih, F. Jasin, M.; Proc Natl Acad Sci USA 1994; 91: 6064-6068). In the absence of a gene targeting vector for homology directed repair, the cells frequently close the double-strand break by non-homologous end-joining (NHEJ). Since this mechanism is error-prone it frequently leads to the deletion or insertion of multiple nucleotides at the cleavage site. If the cleavage site is located within the coding region of a gene it is thereby possible to identify and select mutants that exhibit reading frameshift mutations from a mutagenised population and that represent non-functional knockout alleles of the targeted gene.

In the past, zinc finger nucleases (ZFNs) were developed as a method to apply the stimulatory power of double strand breaks to sequences of endogenous genes, without the need to introduce an artificial nuclease recognition site. Using zinc finger nucleases in the absence of a gene targeting vector for homology directed repair, knockout alleles were generated in mammalian cell lines and knockout zebra fish and rats were obtained upon the expression of ZFN mRNA in one cell embryos (Santiago Y, Chan E, Liu P Q, Orlando S, Zhang L, Urnov F D, Holmes M C, Guschin D, Waite A, Miller J C, Rebar E J, Gregory P D, Klug A, Collingwood T N.; Proc Natl Acad Sci USA 2008; 105:5809-5814; Doyon Y, McCammon J M, Miller J C, Faraji F, Ngo C, Katibah G E, Amora R, Hocking T D, Zhang L, Rebar E J, Gregory P D, Urnov F D, Amacher S L.; Nat Biotechnol 2008; 26:702-708; Geurts A M, Cost G J, Freyvert Y, Zeitler B, Miller J C, Choi V M, Jenkins S S, Wood A, Cui X, Meng X, Vincent A, Lam S, Michalkiewicz M, Schilling R, Foeckler J, Kalloway S, Weiler H, Menoret S, Anegon I, Davis G D, Zhang L, Rebar E J, Gregory P D, Urnov F D, Jacob H J, Buelow R.; Science 2009; 325:433).

Furthermore, zinc finger nucleases were used in the presence of exogenous gene targeting vectors that contain homology regions to the target gene for homology driven repair of the double strand break through gene conversion. This methodology has been applied to gene engineering in mammalian cell lines and gene correction in primary human cells (Urnov F D, Miller J C, Lee Y L, Beausejour C M, Rock J M, Augustus S, Jamieson A C, Porteus M H, Gregory P D, Holmes M C.; Nature 2005; 435:646-651; Porteus M H, Baltimore D. 2003. Science 300:763; Hockemeyer D, Soldner F, Beard C, Gao Q, Mitalipova M, DeKelver R C, Katibah G E, Amora R, Boydston E A, Zeitler B, Meng X, Miller J C, Zhang L, Rebar E J, Gregory P D, Urnov F D, Jaenisch R.; Nat Biotechnol 2009; 27:851-857).

Although the use of zinc finger nucleases results in a higher frequency of homologous recombination, considerable efforts and time are required to design zinc finger proteins that bind a new DNA target sequence at high efficiency. In addition, it has been calculated that using the presently available resources only one zinc finger nuclease could be found within a target region of 1000 base pairs of the mammalian genome (Maeder, et al. 2008 Mol Cell 31(2): 294-301; Maeder, et al. 2009 Nat Protoc 4(10): 1471-501).

The technical problem underlying the present invention is thus the provision of improved means and methods for modifying the genome of eukaryotic cells, such as e.g. mammalian or vertebrate cells.

The solution to this technical problem is achieved by providing the embodiments characterised in the claims.

Accordingly, the present invention relates to a method of modifying a target sequence in the genome of a eukaryotic cell, the method comprising the step: (a) introducing into the cell a fusion protein comprising a DNA-binding domain of a Tal effector protein and a non-specific cleavage domain of a restriction nuclease or a nucleic acid molecule encoding the fusion protein in expressible form, wherein the fusion protein specifically binds within the target sequence and introduces a double strand break within the target sequence.

The term “modifying” as used in accordance with the present invention refers to site-specific genomic manipulations resulting in changes in the nucleotide sequence. The genetic material comprising these changes in its nucleotide sequence is also referred to herein as the “modified target sequence”. The term “modifying” includes, but is not limited to, substitution, insertion and deletion of one or more nucleotides within the target sequence.

The term “substitution”, as used herein, refers to the replacement of nucleotides with other nucleotides. The term includes for example the replacement of single nucleotides resulting in point mutations. Said point mutations can lead to an amino acid exchange in the resulting protein product but may also not be reflected on the amino acid level. Also encompassed by the term “substitution” are mutations resulting in the replacement of multiple nucleotides, such as for example parts of genes, such as parts of exons or introns as well as replacement of entire genes.

The term “insertion” in accordance with the present invention refers to the incorporation of one or more nucleotides into a nucleic acid molecule. Insertion of parts of genes, such as parts of exons or introns as well as insertion of entire genes is also encompassed by the term “insertion”. When the number of inserted nucleotides is not dividable by three, the insertion can result in a frameshift mutation within a coding sequence of a gene. Such frameshift mutations will alter the amino acids encoded by a gene following the mutation. In some cases, such a mutation will cause the active translation of the gene to encounter a premature stop codon, resulting in an end to translation and the production of a truncated protein. When the number of inserted nucleotides is instead dividable by three, the resulting insertion is an “in-frame insertion”. In this case, the reading frame remains intact after the insertion and translation will most likely run to completion if the inserted nucleotides do not code for a stop codon. However, because of the inserted nucleotides, the finished protein will contain, depending on the size of the insertion, one or multiple new amino acids that may effect the function of the protein.

The term “deletion” as used in accordance with the present invention refers to the loss of nucleotides or part of genes, such as exons or introns as well as entire genes. As defined with regard to the term “insertion”, the deletion of a number of nucleotides that is not evenly dividable by three will lead to a frameshift mutation, causing all of the codons occurring after the deletion to be read incorrectly during translation, potentially producing a severely altered and most likely non-functional protein. If a deletion does not result in a frameshift mutation, i.e. because the number of nucleotides deleted is dividable by three, the resulting protein is nonetheless altered as the finished protein will lack, depending on the size of the deletion, several amino acids that may effect the function of the protein.

The above defined modifications are not restricted to coding regions in the genome, but can also occur in non-coding regions of the target genome, for example in regulatory regions such as promoter or enhancer elements or in introns.

Examples of modifications of the target genome include, without being limited, the introduction of mutations into a wild type gene in order to analyse its effect on gene function; the replacement of an entire gene with a mutated gene or, alternatively, if the target sequence comprises mutation(s), the alteration of these mutations to identify which mutation is causative of a particular effect; the removal of entire genes or proteins or the removal of regulatory elements from genes or proteins as well as the introduction of fusion-partners, such as for example purification tags such as the his-tag or the tap-tag etc.

In accordance with the present invention, the term “target sequence in the genome” refers to the genomic location that is to be modified by the method of the invention. The “target sequence in the genome” comprises but is not restricted to the nucleotide(s) subject to the particular modification. Furthermore, the term “target sequence in the genome” also comprises regions for binding of homologous sequences of a second nucleic acid molecule. In other words, the term “target sequence in the genome” also comprises the sequence surrounding the relevant nucleotide(s) to be modified. Preferably, the term “target sequence” refers to the entire gene to be modified.

The term “eukaryotic cell” as used herein, refers to any cell of a unicellular or multi-cellular eukaryotic organism, including cells from animals like vertebrates and from fungi and plants.

The term “fusion protein comprising a DNA-binding domain of a Tal effector protein and a non-specific cleavage domain of a restriction nuclease”, as used in accordance with the present invention, refers to a fusion protein comprising a DNA-binding domain, wherein the DNA-binding domain comprises or consists of Tal effector motifs and the non-specific cleavage domain of a restriction nuclease. The fusion protein employed in the method of the invention retains or essentially retains the enzymatic activity of the native (restriction) endonuclease. In accordance with the present invention, (restriction) endonuclease function is essentially retained if at least 60% of the biological activity of the endonuclease activity are retained. Preferably, at least 75% or at least 80% of the endonuclease activity are retained. More preferred is that at least 90% such as at least 95%, even more preferred at least 98% such as at least 99% of the biological activity of the endonuclease are retained. Most preferred is that the biological activity is fully, i.e. to 100%, retained. Also in accordance with the invention, fusion proteins having an increased biological activity compared to the endogenous endonuclease, i.e. more than 100% activity. Methods of assessing biological activity of (restriction) endonucleases are well known to the person skilled in the art and include, without being limiting, the incubation of an endonuclease with recombinant DNA and the analysis of the reaction products by gel electrophoresis (Bloch K D.; Curr Protoc Mol Biol 2001; Chapter 3:Unit 3.2).

The term “Tal effector protein”, as used herein, refers to proteins belonging to the TAL (transcription activator-like) family of proteins. These proteins are expressed by bacterial plant pathogens of the genus Xanthomonas. Members of the large TAL effector family are key virulence factors of Xanthomonas and reprogram host cells by mimicking eukaryotic transcription factors. The pathogenicity of many bacteria depends on the injection of effector proteins via type III secretion into eukaryotic cells in order to manipulate cellular processes. TAL effector proteins from plant pathogenic Xanthomonas are important virulence factors that act as transcriptional activators in the plant cell nucleus. PthXol, a TAL effector protein of a Xanthomonas rice pathogen, activates expression of the rice gene Os8N3, allowing Xanthomonas to colonize rice plants. TAL effector proteins are characterized by a central domain of tandem repeats, i.e. a DNA-binding domain as well as nuclear localization signals (NLSs) and an acidic transcriptional activation domain. Members of this effector family are highly conserved and differ mainly in the amino acid sequence of their repeats and in the number of repeats. The number and order of repeats in a TAL effector protein determine its specific activity. These repeats are referred to herein as “TAL effector motifs”. One exemplary member of this effector family, AvrBs3 from Xanthomonas campestris pv. vesicatoria, contains 17.5 repeats and induces expression of UPA (up-regulated by AvrBs3) genes, including the Bs3 resistance gene in pepper plants (Kay, et al. 2005 Mol Plant Microbe Interact 18(8): 838-48; Kay, S. and U. Bonas 2009 Curr Opin Microbiol 12(1): 37-43). The repeats of AvrBs3 are essential for DNA binding of AvrBs3 and represent a distinct type of DNA binding domain. The mechanism of sequence specific DNA recognition has been elucidated by recent studies on the AvrBs3, Hax2, Hax3 and Hax4 proteins that revealed the TAL effectors' DNA recognition code (Boch, J., et al. 2009 Science 326: 1509-12).

Tal effector motifs or repeats are 32 to 34 amino acid protein sequence motifs. The amino acid sequences of the repeats are conserved, except for two adjacent highly variable residues (at positions 12 and 13) that determine specificity towards the DNA base A, G, C or T. In other words, binding to DNA is mediated by contacting a nucleotide of the DNA double helix with the variable residues at position 12 and 13 within the Tal effector motif of a particular Tal effector protein (Boch, J., et al. 2009 Science 326: 1509-12).Therefore, a one-to-one correspondence between sequential amino acid repeats in the Tal effector proteins and sequential nucleotides in the target DNA was found. Each Tal effector motif primarily recognizes a single nucleotide within the DNA substrate. For example, the combination of histidine at position 12 and aspartic acid at position 13 specifically binds cytidine; the combination of asparagine at both position 12 and position 13 specifically binds guanosine; the combination of asparagine at position 12 and isoleucine at position 13 specifically binds adenosine and the combination of asparagine at position 12 and glycine at position 13 specifically binds thymidine, as shown in Example 1 below. Binding to longer DNA sequences is achieved by linking several of these Tal effector motifs in tandem to form a “DNA-binding domain of a Tal effector protein”. Thus, the term “DNA-binding domain of a Tal effector protein” relates to DNA-binding domains found in naturally occurring Tal effector proteins as well as to DNA-binding domains designed to bind to a specific target nucleotide sequence as described in the examples below. The use of such DNA-binding domains of Tal effector proteins for the creation of Tal effector motif-nuclease fusion proteins that recognize and cleave a specific target sequence depends on the reliable creation of DNA-binding domains of Tal effector proteins that can specifically recognize said particular target. Methods for the generation of DNA-binding domains of Tal effector proteins are disclosed in the appended examples of this application.

Preferably, the DNA-binding domain is derived from the Tal effector motifs found in naturally occurring Tal effector proteins, such as for example Tal effector proteins selected from the group consisting of AvrBs3, Hax2, Hax3 or Hax4 (Bonas et al. 1989. Mol Gen Genet 218(1): 127-36; Kay et al. 2005 Mol Plant Microbe Interact 18(8): 838-48).

Preferably, the restriction nuclease is an endonuclease. The terms “endonuclease” and “restriction endonuclease” are used herein according to the well-known definitions provided by the art. Both terms thus refer to enzymes capable of cutting nucleic acids by cleaving the phosphodiester bond within a polynucleotide chain. Preferably, the endonuclease is a type II S restriction endonuclease, such as for example FokI, AIwI, SfaNI, SapI, PleI, NmeAIII, MbolI, MlyI, MmeI, HpYAV, HphI, HgaI, FauI, EarI, EciI, BtgZI, CspCI, BspQI, BspMI, BsaXI, BsgI, BseI, BpuEIBmrIBcgIBbvI, BaeI, BbsIAlwI, or AcuI or a type III restriction endonuclease (e.g. EcoP1I, EcoP15I, HinfIII). Also envisaged herein are meganucleases, such as for example I-SceI. More preferably, the endonuclease is FokI endonuclease. FokI is a bacterial type IIS restriction endonuclease. It recognises the non-palindromic penta-deoxyribonucleotide 5′-GGATG-3′: 5′-CATCC-3′ in duplex DNA and cleaves 9/13 nucleotides downstream of the recognition site. FokI does not recognise any specific-sequence at the site of cleavage. Once the DNA-binding domain (either of the naturally occurring endonuclease, e.g. FokI or, in accordance with the present invention, of the fusion protein comprising a DNA-binding domain of a Tal effector protein and a nuclease domain) is anchored at the recognition site, a signal is transmitted to the endonuclease domain and cleavage occurs. The distance of the cleavage site to the DNA-binding site of the fusion protein depends on the particular endonuclease present in the fusion protein. For example, the fusion protein employed in the examples of the present invention cleaves in the middle of a 6 bp sequence that is flanked by the two binding sites of the fusion protein. As a further example, naturally occurring endonucleases such as FokI and EcoP15I cut at 9/13 and 27 bp distance from the DNA binding site, respectively.

Envisaged in accordance with the present invention are fusion proteins that are provided as functional monomers comprising a DNA-binding domain of a Tal effector protein coupled with a single nuclease domain. The DNA-binding domain of a Tal effector protein and the cleavage domain of the nuclease may be directly fused to one another or may be fused via a linker.

The term “linker” as used in accordance with the present invention relates to a sequel of amino acids (i.e. peptide linkers) as well as to non-peptide linkers.

Peptide linkers as envisaged by the present invention are (poly)peptide linkers of at least 1 amino acid in length. Preferably, the linkers are 1 to 100 amino acids in length. More preferably, the linkers are 5 to 50 amino acids in length and even more preferably, the linkers are 10 to 20 amino acids in length. It is well known to the skilled person that the nature, i.e. the length and/or amino acid sequence of the linker may modify or enhance the stability and/or solubility of the molecule. Thus, the length and sequence of a linker depends on the composition of the respective portions of the fusion protein of the invention.

The skilled person is aware of methods to test the suitability of different linkers. For example, the properties of the molecule can easily be tested by testing the nuclease activity as well as the DNA-binding specificity of the respective portions of the fusion protein of the invention.

It will be appreciated by the skilled person that when the fusion protein of the invention is provided as a nucleic acid molecule encoding the fusion protein in expressible form, the linker is a peptide linker also encoded by said nucleic acid molecule.

The term “non-peptide linker”, as used in accordance with the present invention, refers to linkage groups having two or more reactive groups but excluding peptide linkers as defined above. For example, the non-peptide linker may be a polymer having reactive groups at both ends, which individually bind to reactive groups of the individual portions of the fusion protein of the invention, for example, an amino terminus, a lysine residue, a histidine residue or a cysteine residue. The reactive groups of the polymer include an aldehyde group, a propionic aldehyde group, a butyl aldehyde group, a maleimide group, a ketone group, a vinyl sulfone group, a thiol group, a hydrazide group, a carbonyldimidazole (CDI) group, a nitrophenyl carbonate (NPC) group, a trysylate group, an isocyanate group, and succinimide derivatives. Examples of succinimide derivatives include succinimidyl propionate (SPA), succinimidyl butanoic acid (SBA), succinimidyl carboxymethylate (SCM), succinimidyl succinamide (SSA), succinimidyl succinate (SS), succinimidyl carbonate, and N-hydroxy succinimide (NHS). The reactive groups at both ends of the non-peptide polymer may be the same or different. For example, the non-peptide polymer may have a maleimide group at one end and an aldehyde group at another end.

In a preferred embodiment, the linker is a peptide linker.

More preferably, the peptide linker consists of seven glycine residues.

Without wishing to be bound by theory, the present inventors believe that the mechanism of double-strand cleavage by a fusion protein of the invention requires dimerisation of the nuclease domain in order to cut the DNA substrate. Thus, in a preferred embodiment, at least two fusion proteins are introduced into the cell in step (a). Dimerisation of the fusion protein can result in the formation of homodimers if only one type of fusion protein is present or in the formation of heterodimers, when different types of fusion proteins are present. It is preferred in accordance with the present invention that at least two different types of fusion proteins having differing DNA-binding domains of a Tal effector protein are introduced into the cell. The at least two different types of fusion proteins can be introduced into the cell either separately or together. Also envisaged herein is a fusion protein, which is provided as a functional dimer via linkage of two subunits of identical or different fusion proteins prior to introduction into the cell. Suitable linkers have been defined above.

The term “nucleic acid molecule encoding the fusion protein in expressible form” refers to a nucleic acid molecule which, upon expression in a cell or a cell-free system, results in a functional fusion protein. Nucleic acid molecules as well as nucleic acid sequences, as used throughout the present description, include DNA, such as cDNA or genomic DNA, and RNA. Preferably, embodiments reciting “RNA” are directed to mRNA. Furthermore included is genomic RNA, such as in case of RNA of RNA viruses.

It will be readily appreciated by the skilled person that more than one nucleic acid molecule may encode a fusion protein in accordance with the present invention due to the degeneracy of the genetic code. Degeneracy results because a triplet code designates 20 amino acids and a stop codon. Because four bases exist which are utilized to encode genetic information, triplet codons are required to produce at least 21 different codes. The possible 4³ possibilities for bases in triplets give 64 possible codons, meaning that some degeneracy must exist. As a result, some amino acids are encoded by more than one triplet, i.e. by up to six. The degeneracy mostly arises from alterations in the third position in a triplet. This means that nucleic acid molecules having different sequences, but still encoding the same fusion protein can be employed in accordance with the present invention.

In accordance with the present invention, the term “specifically binds within the target sequence and introduces a double strand break within the target sequence” means that the fusion protein is designed such that statistically it only binds to a particular sequence and does not bind to an unrelated sequence elsewhere in the genome. Preferably, the fusion protein in accordance with the present invention comprises at least 18 Tal effector motifs. In other words, the DNA-binding domain of a Tal effector protein within said fusion protein is comprised of at least 18 Tal effector motifs. In the case of fusion proteins consisting of dimers as described above this means that each fusion protein monomer comprises at least nine Tal effector motifs. More preferably, each fusion protein comprises at least 12 Tal effector motifs, such as for example at least 14 or at least 16 Tal effector motifs. Methods for testing the DNA-binding specificity of a fusion protein in accordance with the present invention are known to the skilled person and include, without being limiting, transcriptional reporter gene assays and electrophoretic mobility shift assays (EMSA).

Preferably, the binding site of the fusion protein is up to 500 nucleotides, such as up to 250 nucleotides, up to 100 nucleotides, up to 50 nucleotides, up to 25 nucleotides, up to 10 nucleotides such as up to 5 nucleotides upstream (i.e. 5′) or downstream (i.e. 3′) of the nucleotide(s) that is/are modified in accordance with the present invention.

In a preferred embodiment of the present invention, the modification of the target sequence is by homologous recombination with a donor nucleic acid sequence further comprising the step: (b) introducing a nucleic acid molecule into the cell, wherein the nucleic acid molecule comprises the donor nucleic acid sequence and regions homologous to the target sequence.

The term “homologous recombination”, is used according to the definitions provided in the art. Thus, it refers to a mechanism of genetic recombination in which two DNA strands comprising similar nucleotide sequences exchange genetic material. Cells use homologous recombination during meiosis, where it serves to rearrange DNA to create an entirely unique set of haploid chromosomes, but also for the repair of damaged DNA, in particular for the repair of double strand breaks. The mechanism of homologous recombination is well known to the skilled person and has been described, for example by Paques and Haber (Paques F, Haber J E.; Microbiol Mol Biol Rev 1999; 63:349-404)

In accordance with the present invention, the term “donor nucleic acid sequence” refers to a nucleic acid sequence that serves as a template in the process of homologous recombination and that carries the modification that is to be introduced into the target sequence. By using this donor nucleic acid sequence as a template, the genetic information, including the modifications, is copied into the target sequence within the genome of the cell. In non-limiting examples, the donor nucleic acid sequence can be essentially identical to the part of the target sequence to be replaced, with the exception of one nucleotide which differs and results in the introduction of a point mutation upon homologous recombination or it can consist of an additional gene previously not present in the target sequence.

In accordance with the method of modifying a target sequence of the present invention, the nucleic acid molecule introduced into the cell in step (b) comprises the donor nucleic acid sequence as defined above as well as additional regions that are homologous to the target sequence. It will be appreciated by one of skill in the art that the nucleic acid molecule to be introduced into the cell in step (b) may comprise both the nucleic acid molecule encoding the fusion protein and the nucleic acid molecule comprising the donor nucleic acid sequence and regions homologous to the target sequence. Alternatively, the nucleic acid molecule of step (b) may be a further nucleic acid molecule, to be introduced in addition to the nucleic acid molecule encoding the fusion protein in accordance with step (a).

The term “regions homologous to the target sequence” (also referred to as “homology arms” herein), in accordance with the present invention, refers to regions having sufficient sequence identity to ensure specific binding to the target sequence. Methods to evaluate the identity level between two nucleic acid sequences are well known in the art. For example, the sequences can be aligned electronically using suitable computer programs known in the art. Such programs comprise BLAST (Altschul et al. (1990) J. Mol. Biol. 215, 403), variants thereof such as WU-BLAST (Altschul and Gish (1996) Methods Enzymol. 266, 460), FASTA (Pearson and Lipman (1988) Proc. Natl. Acad. Sci. USA 85, 2444) or implementations of the Smith-Waterman algorithm (SSEARCH, Smith and Waterman (1981) J. Mol. Biol., 147, 195). These programs, in addition to providing a pairwise sequence alignment, also report the sequence identity level (usually in percent identity) and the probability for the occurrence of the alignment by chance (P-value).

Preferably, the “regions homologous to the target sequence” have a sequence identity with the corresponding part of the target sequence of at least 95%, more preferred at least 97%, more preferred at least 98%, more preferred at least 99%, even more preferred at least 99.9% and most preferred 100%. The above defined sequence identities are defined only with respect to those parts of the target sequence which serve as binding sites for the homology arms. Thus, the overall sequence identity between the entire target sequence and the homologous regions of the nucleic acid molecule of step (b) of the method of modifying a target sequence of the present invention can differ from the above defined sequence identities, due to the presence of the part of the target sequence which is to be replaced by the donor nucleic acid sequence.

It is preferred that at least two regions homologous to the target sequence are present in the nucleic acid molecule of (b).

In accordance with the method of the present invention, step (a) of introducing the fusion protein into the cell and step (b) of introducing the nucleic acid molecule into the cell are either carried out concomitantly, i.e. at the same time or are carried out separately, i.e. individually and at different time points. When the steps are carried out concomitantly, both the fusion protein and the nucleic acid molecule can be administered in parallel, for example using two separate injection needles or can be mixed together and, for example, be injected using one needle.

In accordance with the present invention it was surprisingly found that it is possible to introduce gene modifications, including targeted gene modifications, into the genome of eukaryotic cells and to achieve an unexpectedly high frequency of homologous recombination of up to 10% by employing a fusion protein comprising a DNA-binding domain of a Tal effector protein and a non-specific cleavage domain of a restriction nuclease.

Performing the cleavage step of the method of the invention will frequently lead to spontaneous genome modifications through nucleotide loss associated with the repair of double strand breaks by nonhomologous end joining (NHEJ) repair. In addition, by providing a nucleic acid molecule comprising a donor nucleic acid sequence and regions homologous to the target sequence, targeted modification of a genome can be achieved with high specificity.

Several methods are known in the art for achieving an improved frequency of genetic modification. Such methods include, for example, the use of zinc finger nucleases for achieving homologous recombination. However, in order to design zinc finger proteins that bind a new DNA target sequence at high efficiency, considerable efforts and time are required. Furthermore, neighbouring zinc fingers generally influence each other. Thus, they cannot be simply combined into a larger protein in a combinatorial way in order to enhance sequence specificity. As a consequence, the addition of new zinc fingers to a preselected zinc finger protein requires a laborious screening and selection procedure for each individual step. Furthermore, due to the incompletely known DNA binding code and the limited resources of coding zinc finger domains, it is presently difficult to design a nuclease fused to a zinc finger protein specific to any given DNA target sequence. It has been calculated that using the presently available resources only one zinc finger nuclease could be found within a target region of 1000 base-pairs of the mammalian genome (Maeder, et al. 2008 Mol Cell 31(2): 294-301; Maeder, et al. 2009 Nat Protoc 4(10): 1471-501).

Another method employed to achieve a target sequence specific DNA double strand break is the use of yeast derived meganucleases, representing restriction enzymes like I-SceI that binds to specific 18 bp recognition sequence that does not occur naturally in mammalian genomes. However, a combinatorial code for the DNA binding specificity of meganucleases has not been revealed. The redesign of the DNA binding domain of meganucleases allowed so far only the substitution of one or a few nucleotides within their natural binding sequence (Pâques and Duchateau, 2007 Curr Gene Ther 7(1): 49-66). Therefore, the choice of meganuclease target sites is very limited and it is presently not possible to design new meganucleases that bind to any preferred target region within mammalian genomes.

In contrast to these methods, the Tal effector DNA binding domains provide a simple combinatorial code for the construction of new DNA binding proteins with chosen specificity that can be applied to any target sequence within any genome.

In accordance with the present invention a method of introducing genetic modifications into a target genome is provided that overcomes the above discussed problems currently faced by the skilled person. In particular, any number of nucleotide-specific Tal effector motifs can be combined to form a sequence-specific DNA-binding domain to be employed in the fusion protein in accordance with the present invention. Thus, any sequence of interest can now be targeted in a cost-effective, easy and fast way.

In a preferred embodiment, the cells are analysed for successful modification of the target genome.

Methods for analysing for the presence or absence of a modification are well known in the art and include, without being limiting, assays based on physical separation of nucleic acid molecules, sequencing assays as well as cleavage and digestion assays and DNA analysis by the polymerase chain reaction (PCR).

Examples for assays based on physical separation of nucleic acid molecules include without limitation MALDI-TOF, denaturating gradient gel electrophoresis and other such methods known in the art, see for example Petersen et al., Hum. Mutat. 20 (2002) 253-259; Hsia et al., Theor. Appl. Genet. 111 (2005) 218-225; Tost and Gut, Clin. Biochem. 35 (2005) 335-350; Palais et al., Anal. Biochem. 346 (2005) 167-175.

Examples for sequencing assays comprise without limitation approaches of sequence analysis by direct sequencing, fluorescent SSCP in an automated DNA sequencer and Pyrosequencing. These procedures are common in the art, see e.g. Adams et al. (Ed.), “Automated DNA Sequencing and Analysis”, Academic Press, 1994; Alphey, “DNA Sequencing: From Experimental Methods to Bioinformatics”, Springer Verlag Publishing, 1997; Ramon et al., J. Transl. Med. 1 (2003) 9; Meng et al., J. Clin. Endocrinol. Metab. 90 (2005) 3419-3422.

Examples for cleavage and digestion assays include without limitation restriction digestion assays such as restriction fragments length polymorphism assays (RFLP assays), RNase protection assays, assays based on chemical cleavage methods and enzyme mismatch cleavage assays, see e.g. Youil et al., Proc. Natl. Acad. Sci. U.S.A. 92 (1995) 87-91; Todd et al., J. Oral Maxil. Surg. 59 (2001) 660-667; Amar et al., J. Clin. Microbiol. 40 (2002) 446-452.

Alternatively, instead of analysing the cells for the presence or absence of the desired modification, successfully modified cells may be selected by incorporation of appropriate selection markers. Selection markers include positive and negative selection markers, which are well known in the art and routinely employed by the skilled person. Non-limiting examples of selection markers include dhfr, gpt, neomycin, hygromycin, dihydrofolate reductase, G418 or glutamine synthase (GS) (Murphy et al., Biochem J. 1991, 227:277; Bebbington et al., Bio/Technology 1992, 10:169). Using these markers, the cells are grown in selective medium and the cells with the highest resistance are selected. Also envisaged are combined positive-negative selection markers, which may be incorporated into the target genome by homologous recombination or random integration. After positive selection, the first cassette comprising the positive selection marker flanked by recombinase recognition sites is exchanged by recombinase mediated cassette exchange against a second, marker-less cassette. Clones containing the desired exchange cassette are then obtained by negative selection.

In a preferred embodiment of the method of the invention, the cell is selected from the group consisting of a mammalian or vertebrate cell, a plant cell or a fungal cell.

In a further preferred embodiment of the method of the invention, the cell is an oocyte.

As used herein the term “oocyte” refers to the female germ cell involved in reproduction, i.e. the ovum or egg cell. In accordance with the present invention, the term “oocyte” comprises both oocytes before fertilisation as well as fertilised oocytes, which are also called zygotes. Thus, the oocyte before fertilisation comprises only maternal chromosomes, whereas an oocyte after fertilisation comprises both maternal and paternal chromosomes. After fertilisation, the oocyte remains in a double-haploid status for several hours, in mice for example for up to 18 hours after fertilisation.

In a more preferred embodiment of the method of the invention, the oocyte is a fertilised oocyte.

The term “fertilised oocyte”, as used herein, refers to an oocyte after fusion with the fertilizing sperm. For a period of many hours (such as up to 18 hours in mice) after fertilisation, the oocyte is in a double-haploid state, comprising one maternal haploid pronucleus and one paternal haploid pronucleus. After migration of the two pronuclei together, their membranes break down, and the two genomes condense into chromosomes, thereby reconstituting a diploid organism. Preferably, the mammalian or avian oocyte used in the method of the present invention is a fertilised mammalian or avian oocyte in the double-haploid state.

The re-modelling of a fertilised oocyte into a totipotent zygote refers to one of the most complex cell transformations in biology. Remarkably, this transition occurs in the absence of transcription factors and therefore depends on mRNAs accumulated in the oocyte during oogenesis. A growing mouse oocyte, arrested at diplotene of its first meiotic prophase, transcribes and translates many of its own genes, thereby producing a store of proteins sufficient to support development up to the 8-cell stage. These transcripts guide oocytes on the two steps of oocyte maturation and egg activation to become zygotes. Typically, oocytes are ovulated and become competent for fertilisation before reaching a second arrest point. When an oocyte matures into an egg, it arrests in metaphase of its second meiotic division where transcription stops and translation of mRNA is reduced. At this point an ovulated mouse egg has a diameter of 0.085 mm, with a volume of ˜300 picoliter it exceeds 1000-fold the size of a typical somatic cell (Nagy A, Gertsenstein M, Vintersten K, Behringer R., 2003. Manipulating the Mouse Embryo. Cold Spring Harbour, N.Y.: Cold Spring Harbour Laboratory Press).

Life and the embryonic development of a mammal begin when sperm fertilises an egg to form a zygote. Fertilization of the egg triggers egg activation to complete the transformation to a zygote by signaling the completion of meiosis and the formation of pronuclei. At this stage the zygote represents a 1-cell embryo that contains a haploid paternal pronucleus derived from the sperm and a haploid maternal pronucleus derived from the oocyte. In mice this totipotent single cell stage lasts for only ˜18 hours until the first mitotic division occurs.

As totipotent single entities, mammalian zygotes could be regarded as a preferred substrate for genome engineering since the germ line of the entire animal is accessible within a single cell. However, the experimental accessibility and manipulation of zygotes is severely restricted by the very limited numbers at which they are available (dozens-hundred) and their very short lasting nature. These parameters readily explain that the vast majority of genome manipulations, that occur at frequencies of below 10⁻⁵ like gene targeting, can be successfully performed only in cultured embryonic stem cells that are grown up to a number of 10⁷ cells in a single standard culture plate. The only exception from this rule concerns the generation of transgenic mice by pronuclear DNA injection that has been developed into a routine procedure due to the high frequency of transgene integration in up to 30% of injected zygotes (Palmiter R D, Brinster R L.; Annu Rev Genet 1986; 20:465-499). Since microinjected transgenes randomly integrate into the genome, this method can only be used to express additional genes on the background of an otherwise normal genome, but does not allow the targeted modification of endogenous genes.

An early report to characterise the potential of zygotes for targeted gene manipulation by Brinster (Brinster R L, Braun R E, Lo D, Avarbock M R, Oram F, Palmiter R D.; Proc Natl Acad Sci USA 1989; 86:7087-7091), showed that this approach is not practical as only one targeted mouse was obtained from >10.000 zygotes within 14 months of injections. Thus, Brinster et al. discouraged any further attempts in this direction. In addition to a low recombination frequency, Brinster et al. noted a high number of spontaneously occurring, undesired mutations within the targeted allele that severely compromised the function of the (repaired) histocompatibility class II gene. From the experience of Brinster et al. it could be extrapolated that the physiological, biochemical and epigenetic context of genomic DNA in the zygotic pronuclei are unfavourable to achieve targeted genetic manipulations, except for the random integration of transgenes that occurs at high frequency.

In addition, the biology of oocyte development into an embryo provides further obstacles for targeted genetic manipulations. In fertilized mammalian eggs, the two pronuclei that undergo DNA replication, do not fuse directly but approach each other and remain distinct until the membrane of each pronucleus has broken down in preparation for the zygote's first mitotic division that produces a 2-cell embryo. The 1-cell zygote stage is characterised by unique transcriptional and translation control mechanisms. One of the most striking features is a time-dependent mechanism, referred to as the zygotic clock, that delays the expression of the zygotic genome for ˜24 h after fertilization, regardless of whether or not the one-cell embryo has completed S phase and formed a two-cell embryo (Nothias J Y, Majumder S, Kaneko K J, DePamphilis M L.; J Biol Chem 1995; 270:22077-22080). In nature, the zygotic clock provides the advantage of delaying zygotic gene activation (ZGA) until chromatin can be remodelled from a condensed meiotic state to one in which selected genes can be transcribed. Since the paternal genome is completely packaged with protamines that must be replaced with histones, some genes might be prematurely expressed if ZGA were not prevented. Cell-specific transcription requires that newly minted zygotic chromosomes repress most, if not all, promoters until development progresses to a stage where specific promoters can be activated by specific enhancers or trans-activators. In the mouse, formation of a 2-cell embryo marks the transition from maternal gene dependence to zygotic gene activation (ZGA). Among mammals, the extent of development prior to zygotic gene activation (ZGA) varies among species from one to four cleavage events. Maternal mRNA degradation is triggered by meiotic maturation and 90% completed in 2-cell embryos, although maternal protein synthesis continues into the 8-cell stage. In addition to transcriptional control, the zygotic clock delays the translation of nascent mRNA until the 2-cell stage (Nothias J Y, Miranda M, DePamphilis M L.; EMBO J 1996; 15:5715-5725). Therefore, the production of proteins from transgenic expression vectors injected into pronuclei is not achieved until 10-12 hours after the appearance of mRNA.

Geurts et al. have recently found that zinc finger nucleases can be used to induce double strand breaks in the genome of rat zygotes (Geurts A M, Cost G J, Freyvert Y, Zeitler B, Miller J C, Choi V M, Jenkins S S, Wood A, Cui X, Meng X, Vincent A, Lam S, Michalkiewicz M, Schilling R, Foeckler J, Kalloway S, Weiler H, Menoret S, Anegon I, Davis G D, Zhang L, Rebar E J, Gregory P D, Urnov F D, Jacob H J, Buelow R.; Science 2009; 325:433). In this work the induced strand breaks were left for the endogenous, error prone DNA repair mechanism in order to later identify randomly occurring mutant alleles that lost or acquired nucleotides at the site of DNA cleavage. Provided that the zinc finger nuclease cleavage site is located within an exon region of a gene, a reading frame shift will occur in some of the mutant alleles and thereby lead to the production of truncated, non-functional protein. However, this method only leads to the generation of undirected mutations within the coding region of a gene. So far, it has not been possible to induce directed modifications like pre-planned nucleotide substitutions, to insert exogenous DNA sequences like reporter genes and recombinase recognition sites or to replace e.g. murine versus human coding regions.

The introduction of such genetic modifications requires homologous recombination of a specifically designed gene targeting vector with a target gene. Since procedures to achieve high rate homologous recombination in zygotes were not known so far, gene targeting in somatic cells and the subsequent nuclear transfer into enucleated oocytes from sheep and pig have been used as a surrogate technique (Lai L, Prather R S. 2003. Reprod Biol Endocrinol 2003; 1:82; Gong M, Rong Y S. 2003. Curr Opin Genet Dev 13:215-220). However, both techniques are demanding and not very efficient and their combined use is impractical and not well suited for routine application.

In accordance with the present invention a method of introducing genetic modifications into a target genome is provided that overcomes the above discussed problems currently faced by the skilled person. Using the method of the present invention it is now possible to generate genetically modified animals faster, easier and more cost-effective than using any of the prior art methods.

In another preferred embodiment of the method of the invention, the fusion protein or the nucleic acid molecule encoding the fusion protein is introduced into the oocyte by microinjection.

Microinjection into the oocyte can be carried out by injection into the nucleus (before fertilisation), the pronucleus (after fertilisation) and/or by injection into the cytoplasm (both before and after fertilisation). When a fertilised oocyte is employed, injection into the pronucleus is carried out either for one pronucleus or for both pronuclei. Injection of the Tal-finger nuclease or of a DNA encoding the Tal-finger nuclease of step (a) of the method of modifying a target sequence of the present invention is preferably into the nucleus/pronucleus, while injection of an mRNA encoding the Tal-finger nuclease of step (a) is preferably into the cytoplasm. Injection of the nucleic acid molecule of step (b) is preferably into the nucleus/pronucleus. However, injection of the nucleic acid molecule of step (b) can also be carried out into the cytoplasm when said nucleic acid molecule is provided as a nucleic acid sequence having a nuclear localisation signal to ensure delivery into the nucleus/pronucleus. Preferably, the microinjection is carried out by injection into both the nucleus/pronucleus and the cytoplasm. For example, the needle can be introduced into the nucleus/pronucleus and a first amount of the Tal-finger nuclease and/or nucleic acid molecule are injected into the nucleus/pronucleus. While removing the needle from the oocyte, a second amount of the Tal-finger nuclease and/or nucleic acid molecule is injected into the cytoplasm.

Methods for carrying out microinjection are well known in the art and are described for example in Nagy et al. (Nagy A, Gertsenstein M, Vintersten K, Behringer R., 2003. Manipulating the Mouse Embryo. Cold Spring Harbour, N.Y.: Cold Spring Harbour Laboratory Press) as well as in the examples herein below.

In another preferred embodiment of the method of the invention, the nucleic acid molecule of step (b) is introduced into the cell by microinjection.

In a more preferred embodiment, the nucleic acid molecule encoding the fusion protein in expressible form is mRNA.

In another preferred embodiment of the method of the invention, the regions homologous to the target sequence are localised at the 5′ and 3′ end of the donor nucleic acid sequence.

In this preferred embodiment, the donor nucleic acid sequence is flanked by the two regions homologous to the target sequence such that the nucleic acid molecule used in the method of the present invention consists of a first region homologous to the target sequence, followed by the donor nucleic acid sequence and then a second region homologous to the target sequence.

In a further preferred embodiment of the method of the invention, the regions homologous to the target sequence comprised in the nucleic acid molecule have a length of at least 400 bp each. More preferably, the regions each have a length of at least 500 nucleotides, such as at least 600 nucleotides, at least 750 bp nucleotides, more preferably at least 1000 nucleotides, such as at least 1500 nucleotides, even more preferably at least 2000 nucleotides and most preferably at least 2500 nucleotides. The maximum length of the regions homologous to the target sequence comprised in the nucleic acid molecule depends on the type of cloning vector used and can be up to a length 20.000 nucleotides each in E. coli high copy plasmids using the col El replication origin (e.g. pBluescript) or up to a length of 300,000 nucleotides each in plasmids using the F-factor origin (e.g. in BAC vectors such as for example pTARBAC1).

In a further preferred embodiment of the method of the invention, the modification of the target sequence is selected from the group consisting of substitution, insertion and deletion of at least one nucleotide of the target sequence. Preferred in accordance with the present invention are substitutions, for example substitutions of 1 to 3 nucleotides and insertions of exogenous sequences, such as loxP sites (34 nucleotides long) or cDNAs, such as for example for reporter genes. Such cDNAs for reporter genes can, for example, be up to 6 kb long.

In another preferred embodiment of the method of the invention, the cell is from a mammal selected from the group consisting of rodents, dogs, felides, monkeys, rabbits, pigs, or cows or the cell is from an avian selected from the group consisting of chickens, turkeys, pheasants, ducks, geese, quails and ratites including ostriches, emus and cassowaries or the cell is from a fish such a for example zebrafish, salmon, trout, common carp or coi carp.

All of the mammals, avians and fish described herein are well known to the skilled person and are taxonomically defined in accordance with the prior art and the common general knowledge of the skilled person.

Non-limiting examples of “rodents” are mice, rats, squirrels, chipmunks, gophers, porcupines, beavers, hamsters, gerbils, guinea pigs, degus, chinchillas, prairie dogs, and groundhogs.

Non-limiting examples of “dogs” include members of the subspecies canis lupus familiaris as well as wolves, foxes, jackals, and coyotes.

Non-limiting examples of “felides” include members of the two subfamilies: the pantherinae, including lions, tigers, jaguars and leopards and the felinae, including cougars, cheetahs, servals, lynxes, caracals, ocelots and domestic cats.

The term “primates”, as used herein, refers to all monkey including for example cercopithecoid (old world monkey) or platyrrhine (new world monkey) as well as lemurs, tarsiers, apes and marmosets (Callithrix jacchus).

In one embodiment, the mammalian oocyte is not a human oocyte. In another embodiment, the fertilized oocyte is not a human oocyte.

The present invention further relates to a method of producing a non-human vertebrate or mammal carrying a modified target sequence in its genome, the method comprising transferring a cell produced by the method of the invention into a pseudopregnant female host.

In accordance with the present invention, the term “transferring a cell produced by the method of the invention into a pseudopregnant female host” includes the transfer of a fertilised oocyte but also the transfer of pre-implantation embryos of for example the 2-cell, 4-cell, 8-cell, 16-cell and blastocyst (70- to 100-cell) stage. Said pre-implantation embryos can be obtained by culturing the cell under appropriate conditions for it to develop into a pre-implantation embryo. Furthermore, injection or fusion of the cell with a blastocyst are appropriate methods of obtaining a pre-implantation embryo. Where the cell produced by the method of the invention is a somatic cell, derivation of induced pluripotent stem cells is required prior to transferring the cell into a female host such as for example prior to culturing the cell or injection or fusion of the cell with a pre-implantation embryo. Methods for transferring an oocyte or pre-implantation embryo to a pseudo pregnant female host are well known in the art and are, for example, described in Nagy et al., (Nagy A, Gertsenstein M, Vintersten K, Behringer R., 2003. Manipulating the Mouse Embryo. Cold Spring Harbour, N.Y.: Cold Spring Harbour Laboratory Press).

It is further envisaged in accordance with the method of producing a non-human vertebrate or mammal carrying a modified target sequence in its genome that a step of analysis of successful genomic modification is carried out before transplantation into the female host. As a non-limiting example, the oocyte can be cultured to the 2-cell, 4-cell or 8-cell stage and one cell can be removed without destroying or altering the resulting embryo. Analysis for the genomic constitution, e.g. the presence or absence of the genomic modification, can then be carried out using for example PCR or southern blotting techniques or any of the methods described herein above. Such methods of analysis of successful genotyping prior to transplantation are known in the art and are described, for example in Peippo et al. (Peippo J, Viitala S, Virta J, Raty M, Tammiranta N, Lamminen T, Aro J, Myllymaki H, Vilkki J.; Mol Reprod Dev 2007; 74:1373-1378).

Where the cell is an oocyte, the method of producing a non-human vertebrate or mammal carrying a modified target sequence in its genome comprises (a) modifying the target sequence in the genome of a vertebrate or mammalian oocyte in accordance with the method of the invention; (b) transferring the oocyte obtained in (a) to a pseudopregnant female host; and, optionally, (c) analysing the offspring delivered by the female host for the presence of the modification.

For this method of producing a non-human vertebrate or mammal, fertilisation of the oocyte is required. Said fertilisation can occur before the modification of the target sequence in step (a) in accordance with the method of producing a non-human vertebrate or mammal of the invention, i.e. a fertilised oocyte can be used for the method of modifying a target sequence in accordance with the invention. The fertilisation can also be carried out after the modification of the target sequence in step (a), i.e. a non-fertilised oocyte can be used for the method of modifying a target sequence in accordance with the invention, wherein the oocyte is subsequently fertilised before transfer into the pseudopregnant female host.

The step of analysing for the presence of the modification in the offspring delivered by the female host provides the necessary information whether or not the produced non-human vertebrate or mammal carries the modified target sequence in its genome. Thus, the presence of the modification is indicative of said offspring carrying a modified target sequence in its genome whereas the absence of the modification is indicative of said offspring not carrying the modified target sequence in its genome. Methods for analysing for the presence or absence of a modification have been detailed above.

The non-human vertebrate or mammal produced by the method of the invention is, inter alia, useful to study the function of genes of interest and the phenotypic expression/outcome of modifications of the genome in such animals. It is furthermore envisaged, that the non-human mammals of the invention can be employed as disease models and for testing therapeutic agents/compositions. Furthermore, the non-human vertebrate or mammal of the invention can also be used for livestock breeding.

In a preferred embodiment, the method of producing a non-human vertebrate or mammal further comprises culturing the cell to form a pre-implantation embryo or introducing the cell into a blastocyst prior to transferring it into the pseudo pregnant female host. Methods for culturing the cell to form a pre-implantation embryo or introducing the cell into a blastocyst are well known in the art and are, for example, described in Nagy et al., loc. cit.

The term “introducing the cell into a blastocyst” as used herein encompasses injection of the cell into a blastocyst as well as fusion of a cell with a blastocyst. Methods of introducing a cell into a blastocyst are described in the art, for example in Nagy et al., loc. cit.

The present invention further relates to a non-human vertebrate or mammalian animal obtainable by the above described method of the invention.

In a preferred embodiment, the non-human mammal is selected from the group consisting of rodents, dogs, felides, primates, rabbits, pigs, or cows or the vertebrate is selected from the group consisting of fish such as for example zebrafish, salmon, trout, common carp or coi carp or from avians such as for example chickens, turkeys, pheasants, ducks, geese, quails and ratites including ostriches, emus and cassowaries.

The present invention further relates to a fusion protein comprising a Tal effector protein and a non-specific cleavage domain of a restriction nuclease. All the definitions and preferred embodiments defined above with regard to the fusion protein in the context of the method of the invention apply mutatis mutandis. Furthermore, the present invention also relates to a kit comprising the fusion protein of the invention. The various components of the kit may be packaged in one or more containers such as one or more vials. The vials may, in addition to the components, comprise preservatives or buffers for storage. In addition, the kit may contain instructions for use.

The figures show:

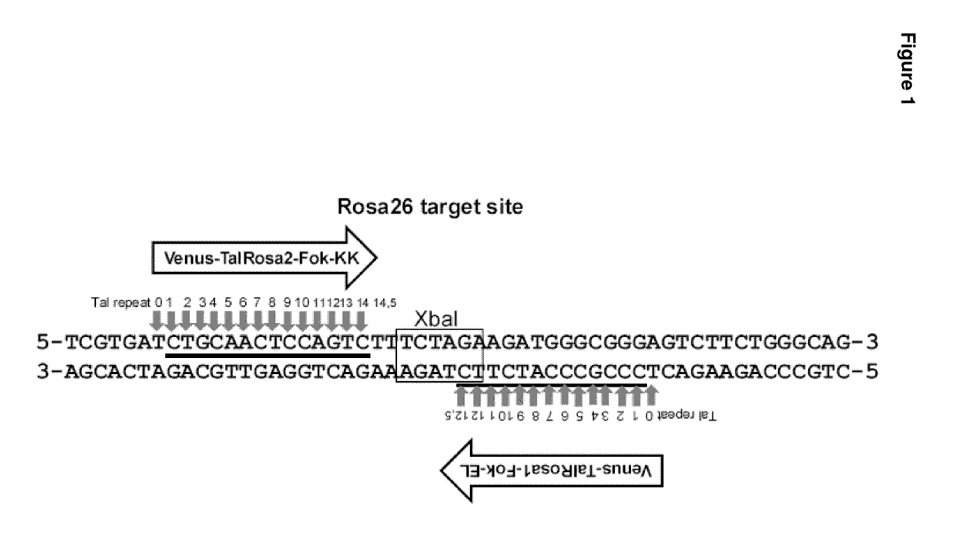

FIG. 1. Design of a fusion protein pair in accordance with the present invention, recognizing the mouse genomic Rosa26 locus. Target sequence from the first intron of the mouse Rosa26 locus containing a central XbaI site. The fusion protein Venus-TalRosa2-Fok-KK contains 14 Tal effector motifs (repeat 1-14) fused to the FokI-KK catalytic domain, recognising the underlined target sequence in the upper DNA strand. Fusion protein Venus-TalRosa1-Fok-EL contains 12 Tal effector motifs (repeat 1-12) that recognize the underlined sequence in the lower DNA strand. Both repeat domains are flanked by the invariable first repeat “0” opposing T and the invariable final repeat “12.5” or “14.5”. The two fusion proteins are separated by a spacer sequence of 6 basepairs.

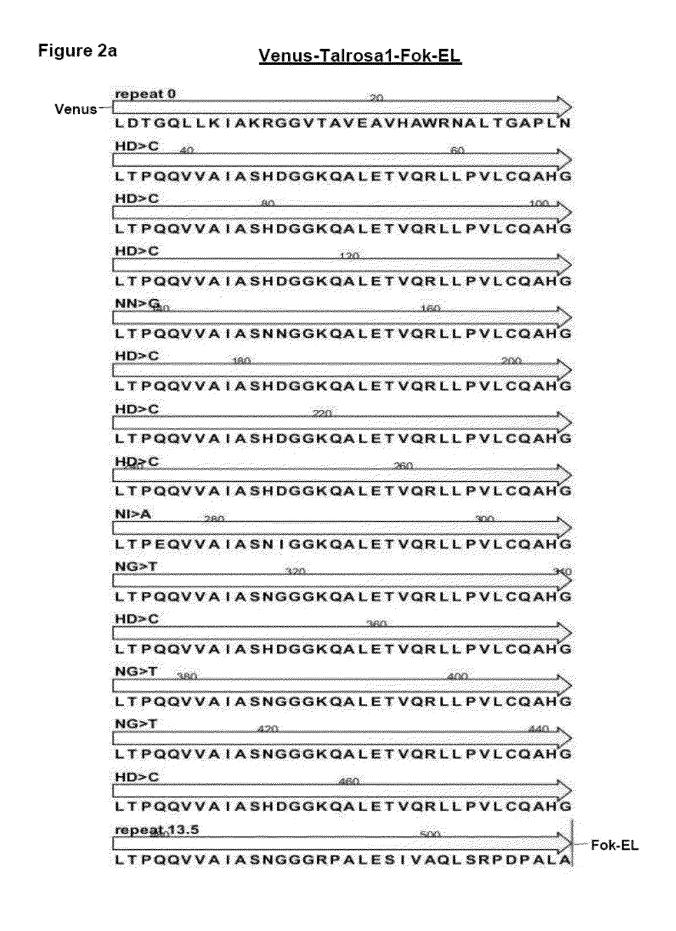

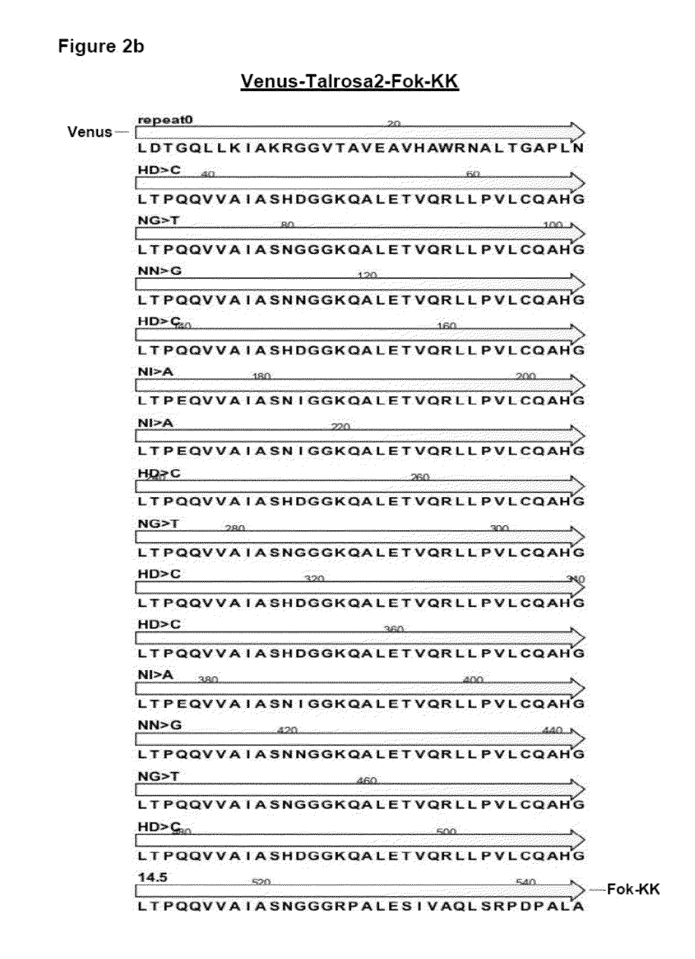

FIG. 2. Structure and amino acid sequence of the fusion proteins of the invention recognizing the mouse genomic Rosa26 locus. Shown is the central part of the pair of Rosa26 specific Tal effector DNA-binding domain—nuclease fusion proteins. Each motif comprises 34 amino acids that vary at positions 12 and 13 and determines specificity towards the Rosa26 target sequence, following the code: H12+D13 recognizing C, N12+N13>G, N12+I13>A and N12+G13>T. Both Tal effector DNA-binding domains are N-terminally fused to Venus and C-terminally fused to the FokI catalytic variant domain Fok-KK or Fok-EL.

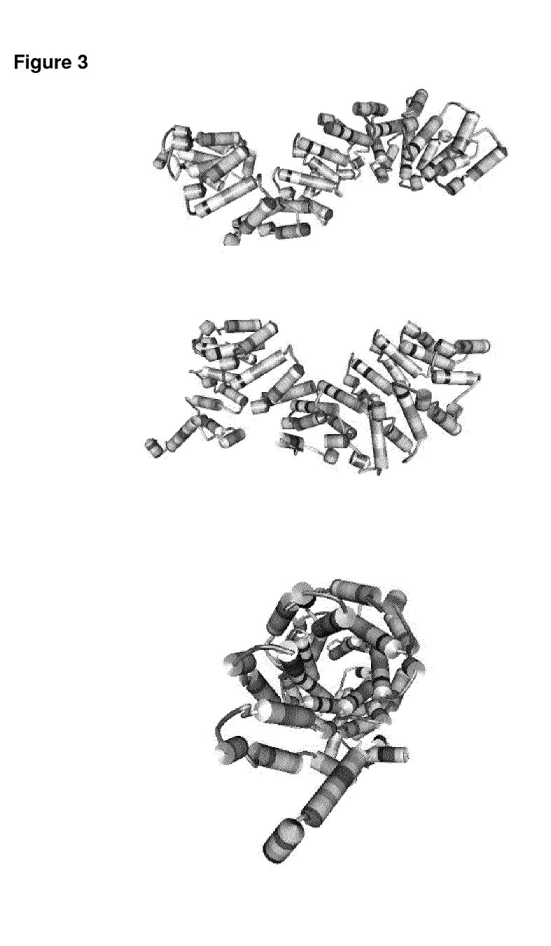

FIG. 3. Structural model of a Tal effector DNA-binding domain—nuclease fusion protein of the invention. Structural modeling of an array of 14 Tal effector motifs recognizing a target sequence (GGT-GGC-CCG-GTA-GT) within the mouse Rab38 gene, using the I-Tasser software. As seen in the top (upper graph) and bottom views (middle graph) the Tal effector motifs array in a superhelical structure that could surround a central DNA molecule (not shown). Accordingly, the side view (bottom graph) reveals a free central space to accommodate a substrate DNA molecule. Protein regions forming alpha-helices are shown as schematic tubes; each 34 residue Tal effector motif folds into two helices that are connected by the exposed amino acids at position 12 and 13 that determine DNA sequence specific binding.

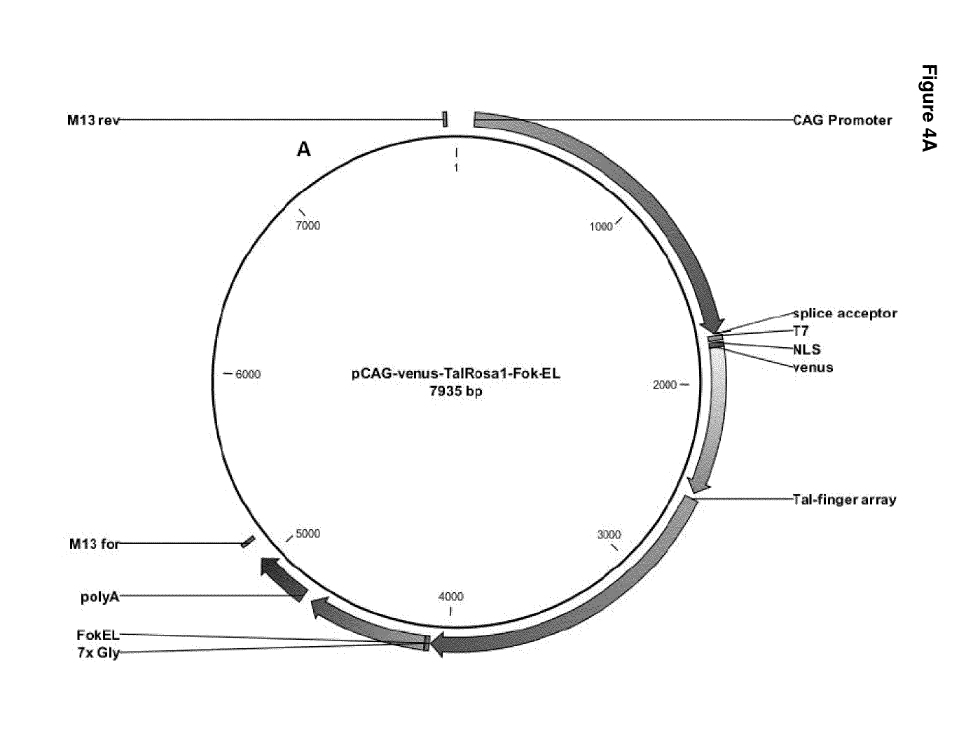

FIG. 4. Expression vectors for Tal effector DNA-binding domain—nuclease fusion proteins of the invention. The Rosa26 target sequence specific Tal effector DNA-binding domains TalRosa1 and Talrosa2 are ligated in frame into a plasmid backbone that provides a N-terminal fusion with Venus (including a nuclear localisation signal—NLS) and a C-terminal fusion with the KK or EL mutant of FokI nuclease, to derive the plasmid pCAG-venus-TalRosa1-Fok-EL (SEQ ID No:2) and pCAG-venus-TalRosa2-Fok-KK (SEQ ID No:4). The Tal effector DNA-binding domain is connected to the Fok domain by a peptide linker of seven glycine residues (7×Gly). The coding region of the venus-TalRosa-Fok proteins can be transcribed in vertebrate cells into mRNA from the CAG hybrid promoter and terminated by a polyadenylation signal sequence (polyA) derived from the bovine growth hormone gene. Alternatively mRNA can be transcribed in vitro from the phage derived T7 promoter located upstream of the ATG start codon and translated in vitro into the venus-TalRosa1-Fok-EL (SEQ ID No:3) and venus-TalRosa2-Fok-KK (SEQ ID No:5) proteins.

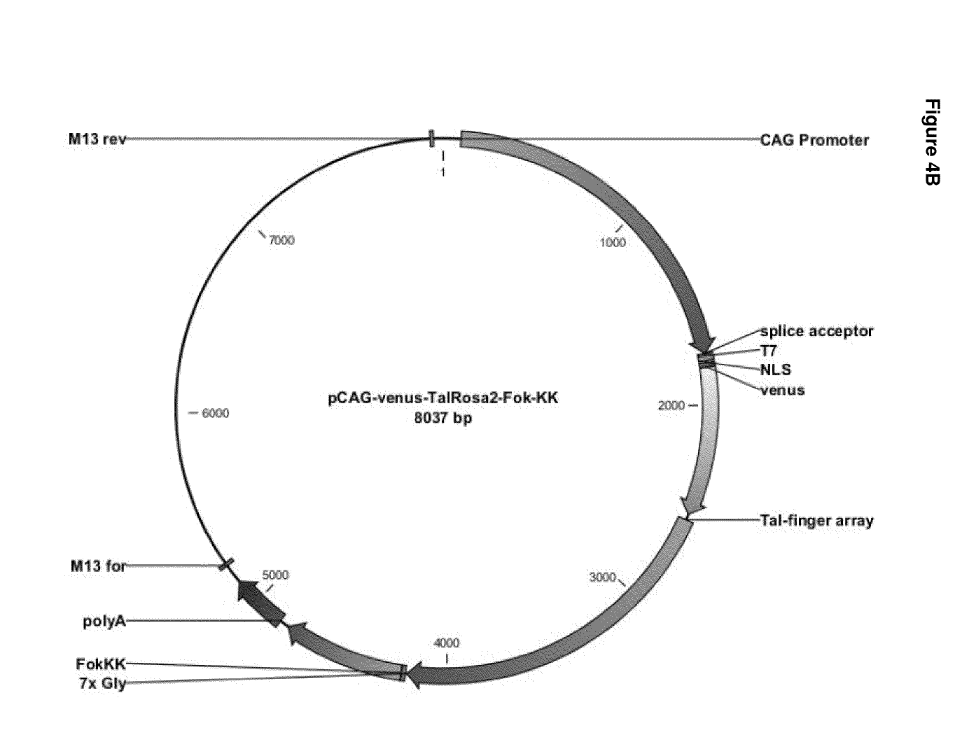

FIG. 5. Gene targeting vector pRosa26.8-2 and Tal effector DNA-binding domain—nuclease-assisted homologous recombination at the mouse Rosa26 locus. A: Structure of the gene targeting vector pRosa26.8-2. The 5′ and 3′ homology regions (5′HR, 3′HR) to the Rosa26 locus are flanking a reporter gene cassette comprising a splice acceptor (SA) sequence, the β-galactosidase coding region and a polyadenylation sequence (pA); B: Genomic structure of the mouse Rosa26 locus. Shown are the first 2 exons of Rosa26 and the Rosa26 promoter (arrow) upstream of exon 1. The homology regions to the pRosa26.8-2 vector within intron 1 are indicated by stippled lines and the target site for the pair of Tal effector DNA-binding domain—nuclease fusion proteins (FIG. 1, FIG. 2) is shown by an arrow. Upon a fusion protein-induced double strand break at the target site, homologous recombination with pRosa26.8 is stimulated resulting in a recombined Rosa26 locus; C: Recombined Rosa26 locus. Upon recombination mediated transfer of the reporter gene cassette into the target site for the fusion protein the reporters splice acceptor is spliced to the Rosa26 exon 1 sequence, leading to the production of a mRNA coding for β-galactosidase (βGal.).

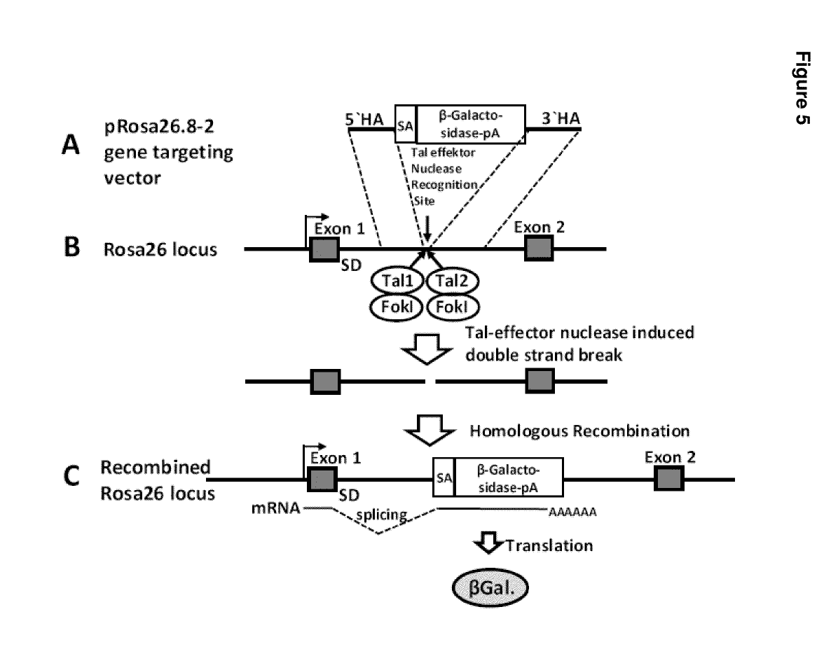

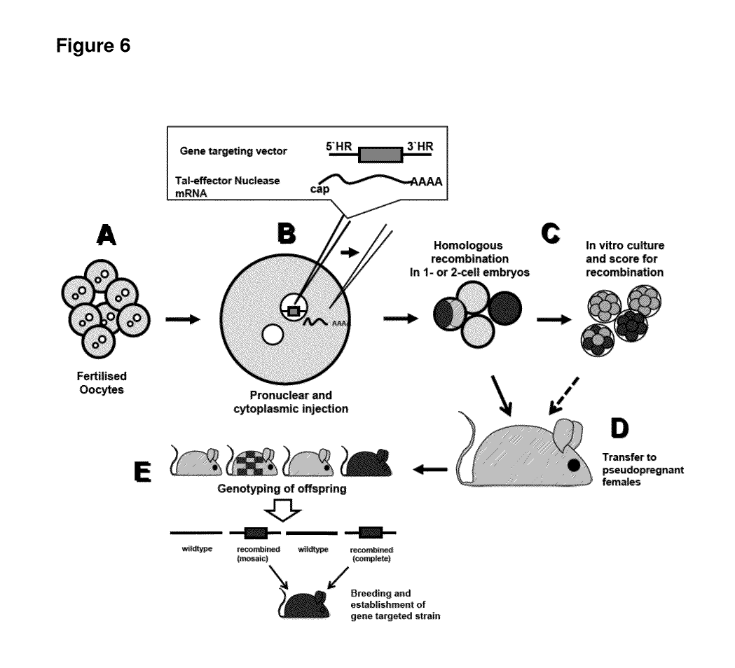

FIG. 6. Scheme for the generation of genetically modified mice at the Rosa26 locus by injection of the pRosa26.8-2 gene targeting vector together with mRNA coding for Rosa26 specific fusion protein. A: Fertilised oocytes, collected from superovulated females; B: Microinjection of a gene targeting vector and mRNA coding for Tal effector DNA-binding domain—nuclease fusion proteins into one pronucleus and the cytoplasm of a fertilised oocyte; C: In vitro culture of injected embryos and assessment of reporter gene activity. Injected embryos can either directly transferred to pseudopregnant females or after detection of the reporter activity if a live stain is used; D: Pseudopregnant females deliver live offspring from microinjected oocytes, E: The offspring is genotyped for the presence of the induced genetic modification. Positive animals are selected for further breeding to establish a gene targeted strain.

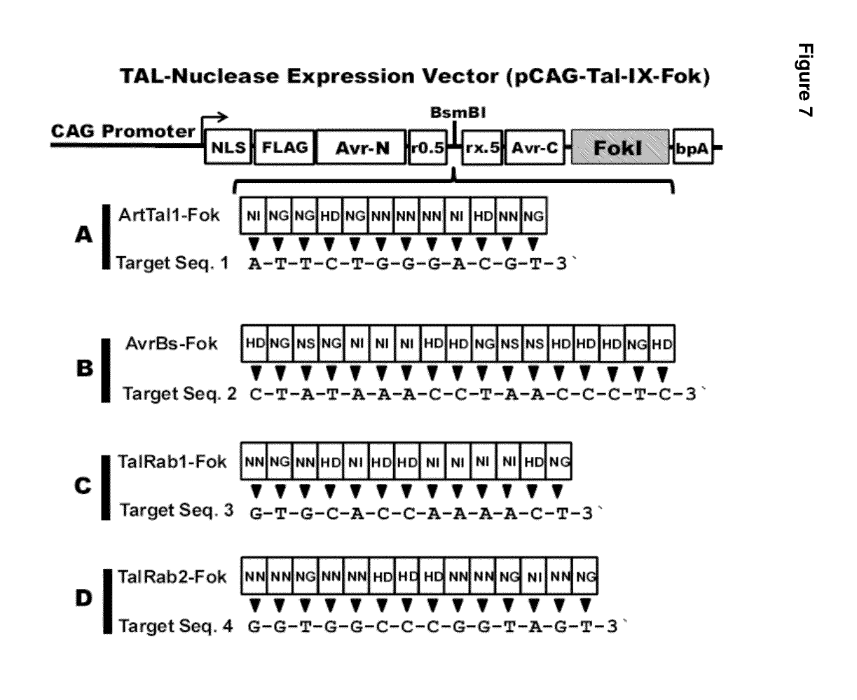

FIG. 7: TAL-FokI Nuclease Expression Vectors

The Tal nuclease expression vector pCAG-Tal-IX-Fok contains a CAG promoter region and a transcriptional unit comprising, upstream of a central pair of BsmBI restriction sites, an ATG start codon (arrow), a nuclear localisation sequence (NLS), a FLAG Tag sequence (FLAG), a linker, a segment coding for 110 amino acids of the Tal protein AvrBs3 (AvrN) and its invariable N-terminal Tal repeat (r0.5). Downstream of the BsmBI sites the transcriptional unit contains an invariable C-terminal Tal repeat (rx.5), a segment coding for 44 amino acids derived from the Tal protein AvrBs3, the coding sequence of the FokI nuclease domain and a polyadenylation signal sequence (bpA). DNA segments coding for Tal repeats can be inserted into the BsmBI sites of pCAG-Tal-IX-Fok for the expression of variable Tal-Fok nuclease fusion proteins. A: to create the ArtTal1-Fok Tal nuclease an array of 12 Tal repeats recognising the indicated target sequence #1 was inserted into pCAG-Tal-IX-Fok. B: to create the AvrBs-Fok Tal nuclease an array of 17 Tal repeats recognising the indicated target sequence #2 was inserted into pCAG-Tal-IX-Fok. C: to create the TalRab1-Fok Tal nuclease an array of 13 Tal repeats recognising the indicated target sequence #3 was inserted into pCAG-Tal-IX-Fok. D: to create the TalRab2-Fok Tal nuclease an array of 14 Tal repeats recognising the indicated target sequence #4 was inserted into pCAG-Tal-IX-Fok. Each 34 amino acid Tal repeat is drawn as a square indicating the repeat's amino acid code at positions 12/13 that confers binding to one of the DNA nucleotides of the target sequence (NI>A or NS>A, NG >T, HD>C, NN>G) shown below.

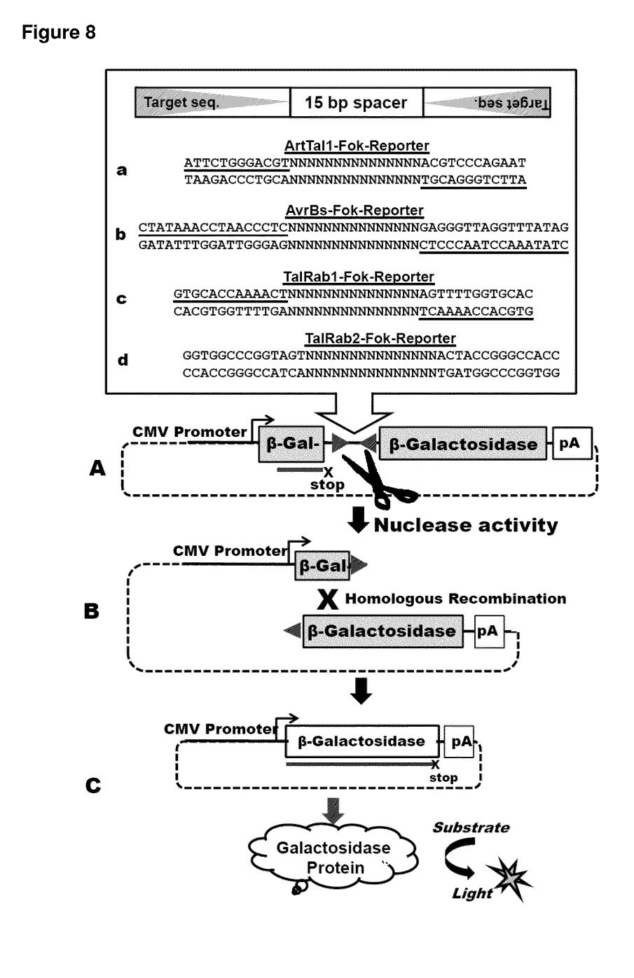

FIG. 8: Tal Nuclease Reporter Assay

A: Tal nuclease reporter plasmids contain a CMV promoter region, a 400 bp sequence coding for the N-terminal segment of β-galactosidase and a stop codon. This unit is followed by a Tal nuclease target region consisting of two inverse oriented recognition sequences (underlined) for ArtTal-Fok (a), AvrBs-Fok (b), TalRab1-Fok (c), or TalRab2-Fok (d) that are separated by a 15 bp spacer region (NNN . . . ). The Tal nuclease target region is followed by the complete coding region for β-galactosidase and a polyadenylation signal (pA). To test for nuclease activity against the target sequence a Tal nuclease expression vector (FIG. 7) is transiently cotransfected with its corresponding reporter plasmid into HEK 293 cells. Upon expression of the Tal nuclease protein the reporter plasmid is opened by a nuclease induced double strand-break within the Tal nuclease target sequence (scissor). B: The DNA regions adjacent to the double-strand break are identical over 400 bp and can be aligned and recombined (X) by homologous recombination DNA repair. C: Homologous recombination of an opened reporter plasmid results into a functional β-galactosidase expression vector that produces the β-galactosidase enzyme. After two days the transfected cell population is lysed and the enzyme activity in the lysate is determined by a chemiluminescent reporter assay. The levels of the reporter catalysed light emission are measured and indicate Tal nuclease activity.

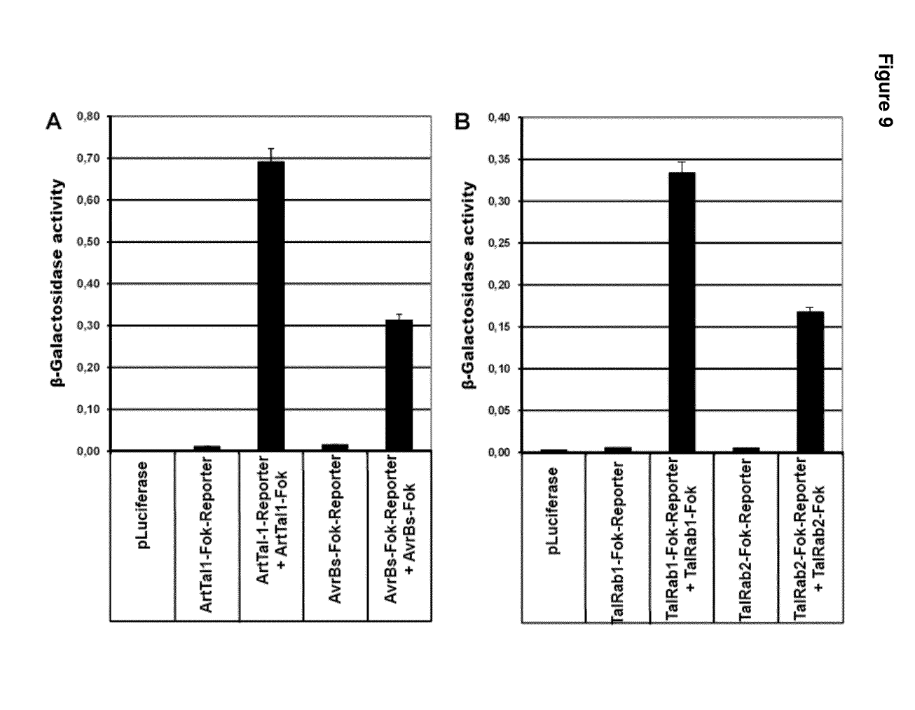

FIG. 9: Activity of Tal Nucleases in HEK 293 Cells

To test for the nuclease activity of Tal nucleases, expression vectors for ArtTal1-Fok, AvrBs-Fok, TalRab1-Fok and TalRab2-Fok (FIG. 7) were transiently transfected together with the corresponding reporter plasmids (FIG. 8) into HEK 293 cells. Specific nuclease activity against the reporter plasmid's target sequence leads to homologous recombination and the expression of β-galactosidase. Two days after transfection the cell populations were lysed and the β-galactosidase activity was determined by a chemiluminescent reporter assay. The levels of light emission were normalised in relation to the activity of a cotransfected Luciferase expression plasmid and are shown in comparison to the activity of the positive control β-galactosidase vector pCMVβ, that was defined as 1.0. The values for each transfected sample represent the mean value and SD derived from three culture wells transfected side by side. A: The transfection of the ArtTal1-Fok or AvrBs-Fok-Reporter plasmids without nuclease expression vectors results in a low background level of β-galactosidase, comparable to the transfection of the Luciferase plasmid alone. In contrast, the cotransfection of pCAG-ArtTal1-Fok with ArtTal1-Fok-Reporter plasmid or of pCAG-AvrBs-Fok with AvrBs-Fok-Reporter plasmid resulted in a strong increase of β-galactosidase activity, indicating the nuclease activity of the Tal nucleases ArtTal1-Fok and AvrBs-Fok. B: The transfection of the TalRab1-Fok or TalRab2-Fok reporter plasmids without nuclease expression vectors results in a low background level of β-galactosidase, comparable to the transfection of the Luciferase plasmid alone. In contrast, the cotransfection of pCAG-TalRab1-Fok with TalRab1-Fok-Reporter plasmid or of pCAG-TalRab2-Fok with TalRab2-Fok-Reporter plasmid resulted in a 30-50-fold increase of β-galactosidase activity, indicating the nuclease activity of the Tal nucleases TalRab1-Fok and TalRab2-Fok.

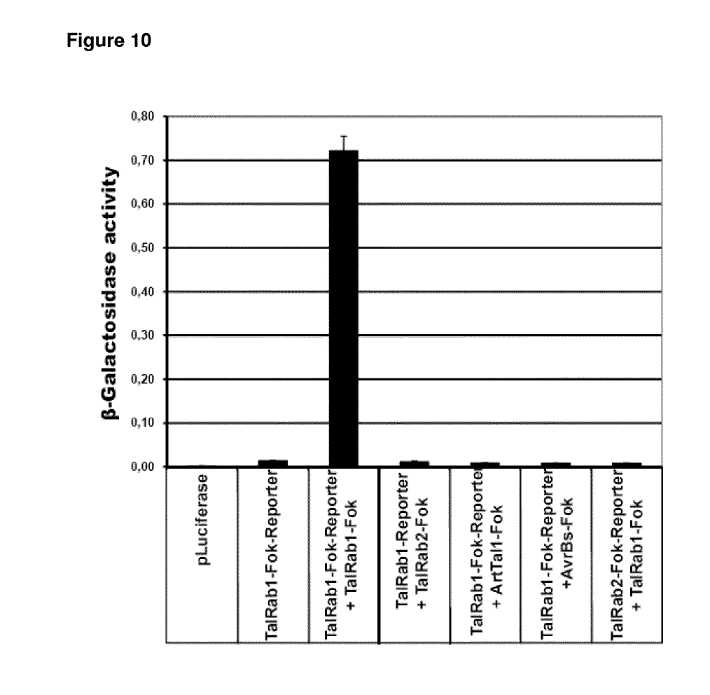

FIG. 10: Target Sequence Specificity of Tal Nucleases

To test for the specificity of the TalRab1-Fok and TalRab2-Fok nucleases against their predicted target sequence in comparison to an unrelated DNA sequence, the TalRab1-Fok-Reporter plasmid was transfected alone, cotransfected with the corresponding expression vector for TalRab1-Fok, or together with the expression vectors for TalRab2-Fok, ArtTal1-Fok or AvrBs-Fok. Strong nuclease activity developed only in the specific combination of the ArtTal1-Fok expression vector together with the ArtTal1-Fok-Reporter plasmid. Vice versa the TalRab1-Fok expression vector did not exhibit nuclease activity against the TalRab2-Fok-Reporter plasmid.

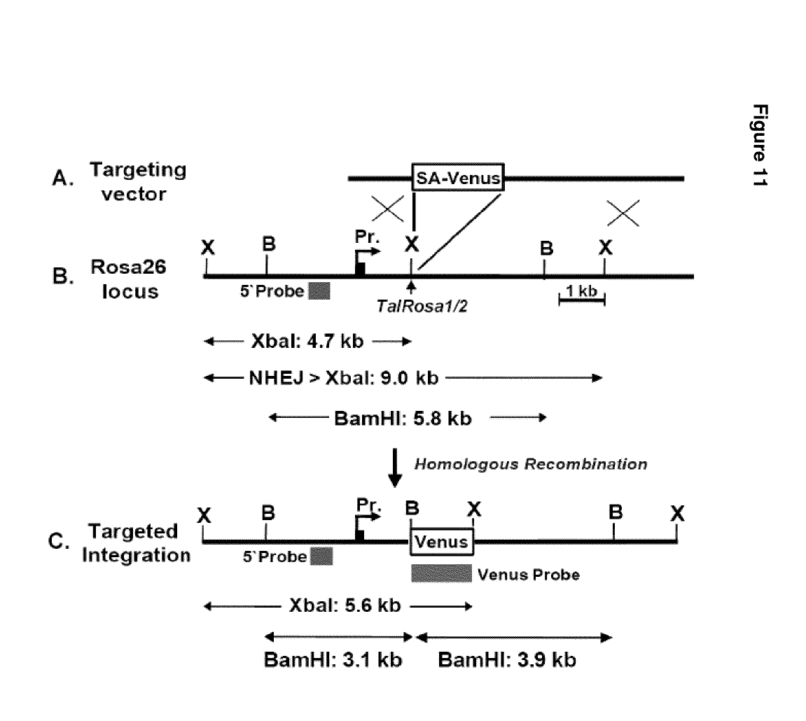

FIG. 11: Targeted Integration of a Venus Reporter Gene into the Rosa26 Locus.

A: Targeting vector pRosa26.3-3 for insertion of a 1.1 kb Venus gene, including a splice acceptor (SA) and polyA site, into the Rosa26 locus. The location of the Rosa26 promoter (Pr.), first exon, of the Rosa-5′ and venus Southern blot probes and XbaI (X) and BamHI (B) sites and fragments are indicated. B: Structure of the Rosa26 wildtype locus, including the TAL-nuclease recognition sites that overlap with an intronic XbaI site (X). C: Structure of the recombined Rosa26 allele. The wildtype Rosa26 locus exhibits a 5.8 kb BamHI band, whereas targeted integration of the reporter gene is indicated by the presence of a predicted 3.1 kb BamHI fragment detected with the Rosa26 5′-probe. The targeted locus exhibits a 3.9 kb band using the venus hybridization probe.

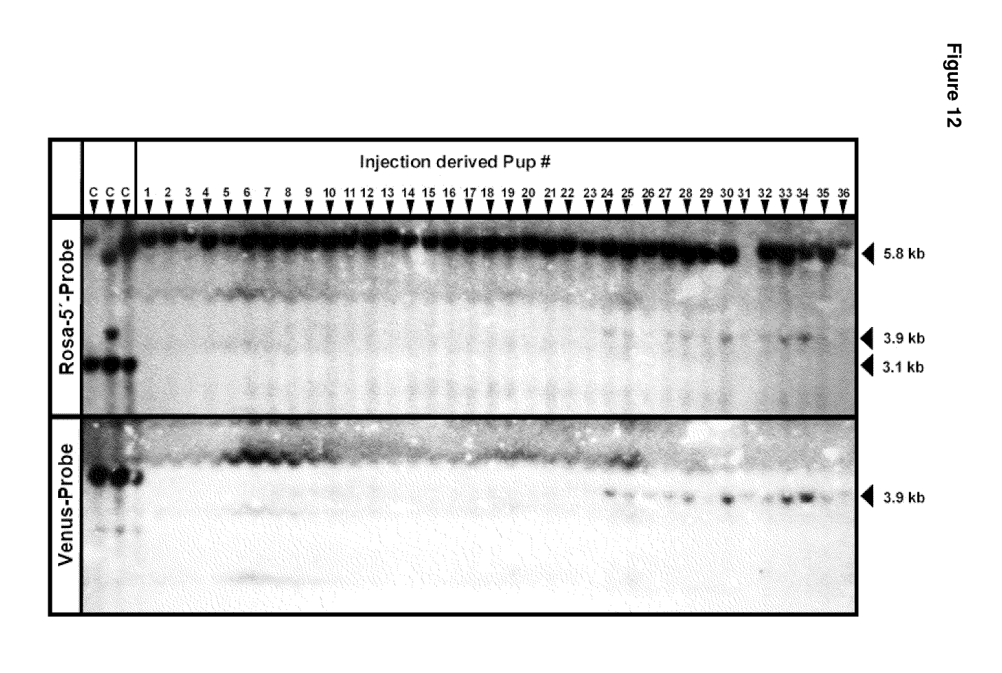

FIG. 12: Targeted Integration of a Venus Reporter Gene into the Rosa26 Locus.

Genomic tail DNA of mice derived from zygote coinjections of TalRosa1, TalRosa2 mRNA and targeting vector pRosa26.3-3 was digested with BamHI and analyzed by Southern blotting using the Rosa26 5′-probe (upper box) or the venus probe (lower box). The analysis of BamHI digested DNA with the internal Venus probe showed the predicted 3.9 kb band in the samples #24-28 and #30-34. The analysis of BamHI digested DNA with the Rosa26 5′-probe showed the 5.8 kb wildtype band and an additional band, indicating recombination at Rosa26, in samples #24-28, #30, and 32-34. This additional band appeared at a size of 3.9 kb instead of the predicted 3.1 kb fragment. Three lanes labeled with “C” show BamHI digestions of tail DNA from control mice that contain the Rosa26.3-3 targeted allele (FIG. 11C) in their germline.

The examples illustrate the invention.

### EXAMPLE 1

### Construction of Rosa26 Specific Tal Effector DNA-Binding Domain—Nuclease Fusion Proteins

Fusion Protein Design

To demonstrate the functionality of Tal effector DNA-binding domain—nuclease fusion proteins in mammalian cells we designed a pair of fusion proteins that recognizes a DNA target sequence within the mouse Rosa26 locus (FIG. 1) (SEQ ID NO: 1). The two Tal effector DNA-binding domain—nuclease fusion proteins are intended to bind together to the bipartite target DNA region and to induce a double strand break in the spacer region of the target region to stimulate homologus recombination at the target locus in mammalian cells. The Rosa26 target nucleotides were selected such that the binding regions of the fusion proteins are separated by a spacer of 6 basepairs and each target sequence is preceeded by a T. Following the sequence downstream of the initial T in the 5′>3′ direction, base specific Tal effector DNA-binding domain—nuclease fusion proteins were combined together in a N to C terminal order into an array of 12 (TalRosa1) or 14 Tal-fingers (TalRosa2), preceeded by a invariable first (0) and last Tal-finger (12,5; 14,5) (FIG. 1). Each Tal effector motif consists of 34 amino acids the position 12 and 13 of which determines the specificity towards recognition of A, G, C or T within the target sequence (Boch, J., et al. 2009 Science 326: 1509-12). To derive Rosa26 specific Tal effector DNA-binding domain—nuclease fusion proteins (FIG. 2) we selected the Tal effector motif (repeat) #11 derived from the Xanthomonas Hax3 protein (GenBank accession No. AY993938.1 (LTPEQVVAIASNIGGKQALETVQRLLPVLCQAHG; SEQ ID NO: 24) with amino acids N12 and 113 to recognize A, the Tal effector motif (repeat) #5 (LTPQQVVAIASHDGGKQALETVQRLLPVLCQAHG; SEQ ID NO: 25) derived from the Hax3 protein with amino acids H12 and D13 to recognize C, and the Tal effector motif (repeat) #4 (LTPQQVVAIASNGGGKQALETVQRLLPVLCQAHG; SEQ ID NO: 26) from the Xanthomonas Hax4 protein (Genbank accession No.: AY993939.1) with amino acids N12 and G13 to recognize T. To recognize a target G nucleotide we used the Tal effector motif (repeat) #4 from the Hax4 protein with replacement of the amino acids 12 into N and 13 into N (LTPQQVVAIASNNGGKQALETVQRLLPVLCQAHG; SEQ ID NO: 27). The base specific DNA-binding domains are preceeded by the invariable first Tal-repeat (LDTGQLLKIAKRGGVTAVEAVHAWRNALTGAPLN; SEQ ID NO: 28) and followed by the last Tal-repeat (LTPEQVVAIASNGGGRPALESIVAQLSRPDPALA; SEQ ID NO: 29) from the Hax3 protein. The DNA-binding domains of the Tal effector proteins recognizing the Rosa26 target sequence were designed in silico using the Vector NTI (Invitrogen) or DNA workbench (CLC) software and combined in frame N-terminally with the GFP variant Venus and C-terminally, via a linker peptide of 7 glycine resiues, with the catalytic domain of FokI endonuclease to derive the pair of Tal effector DNA-binding domain—nuclease fusion proteins, i.e. venus-TalRosa1-Fok-EL (SEQ ID NO:3) and venus-TalRosa2-Fok-KK (SEQ ID NO:5) (FIG. 2). The catalytic domain of FokI endonuclease normally acts as a homodimer. To avoid the homodimer formation of a single TalRosa nuclease at nonintended genomic target sequences and thereby to increase the specificity of the Tal effector DNA-binding domain—nuclease fusion protein pair, we used the FokI mutant domains “KK” and “EL” that preferentially act only as heterodimer (Miller et al. 2007 Nat Biotechnol 25(7): 778-85). In order to model the binding of the fusion proteins of the invention to a DNA target sequence we calculated the 3D structure of a 14 Tal effector motif protein designed to recognize the sequence 5′-GGTGGCCCGGTAGT-3′ within the mouse Rab38 gene using the 1-Tasser software (Roy et al. 2010 Nat Protoc 5(4): 725-38) and visualized the structure using the Discovery studio software (Accelerys) (FIG. 3). According to this structural model the Tal effector motifs fold into a superhelical structure prepared to accomodate a central DNA molecule. Each 34 residue Tal effector motif folds into two helices that are connected by the exposed amino acids at position 12 and 13 that determine DNA sequence specific binding (FIG. 3).

Expression Vectors

To derive vectors for the expression of Tal effector DNA-binding domain—nuclease fusion proteins in mammalian cells the Rosa26 specific coding regions for the Tal effector DNA-binding domain were synthesized by a commercial service provider (Geneart, Regensburg, Germany). The coding DNA fragments for the Tal effector DNA-binding domains TalRosa1 and Talrosa2 were ligated in frame into a plasmid backbone that provides elements for mRNA and protein expression in mammalian cells, specifically a N-terminal fusion with the Venus fluorescent protein (including a nuclear localisation signal—NLS) and a C-terminal fusion with the KK or EL mutant of FokI nuclease, to derive the plasmids pCAG-venus-TalRosal-Fok-EL (SEQ ID NO: 2) and pCAG-venus-TalRosa2-Fok-KK (SEQ ID NO: 4). The Tal effector DNA-binding domain is connected to the Fok domain by a peptide linker of seven glycine residues (7×Gly). The coding region of the venus-TalRosa-Fok proteins can be transcribed in mammalian cells into mRNA from the CAG hybrid promoter and terminated by a polyadenylation signal sequence (polyA) derived from the bovine growth hormone gene. Alternatively mRNA can be transcribed in vitro from the phage derived T7 promoter located upstream of the ATG start codon and translated in vitro into the venus-TalRosa1-Fok-EL (SEQ ID NO: 3) and venus-TalRosa2-Fok-KK (SEQ ID NO: 5) proteins.

DNA Cleavage Activity of Tal Effector DNA-Binding Domain—Nuclease Fusion Proteins

The designed Tal effector DNA-binding domain—nuclease fusion proteins are tested for function by an in vitro nuclease cleavage assay. For this purpose mRNA and protein of the venus-TalRosa-Fok nuclease fusion proteins are produced from the pCAG-venus-TalRosal-Fok-EL and pCAG-venus-TalRosa2-Fok-KK plasmids using the TnT Quick coupled in vitro transcription/translation system from Promega (Madison, Wis., USA) following the manufacturers instructions. In an in vitro nuclease assay (Kandavelou 2009 Methods Mol Biol 544: 617-36) a fraction of the synthesized proteins is incubated together with the plasmid pbs-Rosa-targetseq (SEQ ID NO: 7) that contains the Rosa26 target sequence, to assess the cleavage activity of the Tal effector DNA-binding domain—nuclease fusion protein pair. The reaction is analysed for cleavage of the DNA substrate by agarose gel electrophoresis and reveals that the Tal-finger nuclease pair can induce a double strand break within the Rosa26 target sequence.

### EXAMPLE 2

### Tal Effector DNA-Binding Domain—Nuclease Fusion Protein-Assisted Homologous Recombination in Fertilized Mouse Oocytes

With this experiment it is tested whether homologous recombination at the site of a double strand break induced by a Tal effector DNA-binding domain—nuclease fusion protein occurs in fertilised mouse oocytes at a reasonable frequency (>1%). For this purpose we constructed the gene targeting vector pRosa26.8-2 (SEQ ID NO: 6) that inserts a reporter gene cassette into the mouse Rosa26 locus via homology regions. This vector comprises a splice acceptor element, the coding region of β-galactosidase and a polyadenylation sequence, combined with a 1 kb 5-and 4 kb 3′-homology region derived from the first intron of the Rosa26 locus (FIG. 5A, B). The Rosa26 locus is a region on chromosome 6 that has been found to be ubiquitously expressed in all tissues and developmental stages of the mouse and is suitable for transgene expression (Zambrowicz B P, Imamoto A, Fiering S, Herzenberg L A, Kerr W G, Soriano P.; Proc Natl Acad Sci USA 1997; 94:3789-3794; Seibler J, Zevnik B, Kuter-Luks B, Andreas S, Kern H, Hennek T, Rode A, Heimann C, Faust N, Kauselmann G, Schoor M, Jaenisch R, Rajewsky K, Kuhn R, Schwenk F.; Nucleic Acids Res 2003; 31:e12.). Upon recombination the vector splice acceptor is spliced to the donor site of the Rosa26 transcript such that the fusion transcript codes for β-galactosidase (FIG. 5C).

A) Results

The linearised targeting vector is microinjected into fertilised mouse oocytes (FIG. 6A, B) together with in vitro transcribed mRNA coding for the pair of Tal effector DNA-binding domain—nuclease fusion proteins (FIG. 2) that recognise the target sequence of Rosa26 (FIG. 1) and induce a double strand break at the insertion site of the reporter gene cassette (FIG. 5B). Upon microinjection, the Tal effector DNA-binding domain—nuclease fusion protein mRNAs are translated into proteins that induce a double strand break at one or both Rosa26 alleles in one or more cells of the developing embryo. This event stimulates the recombination of the pRosa26.8-2 vector with a Rosa26 allele via the homology regions present in the vector and leads to the site-specific insertion of the non-homologous reporter gene cassette into the genome (FIG. 5C). Depending on the timing of these events recombination may occur within the one cell embryo or later in only a single cell of a 2-cell, 4-cell or 8-cell embryo. To detect such successful recombination events the microinjected zygotes are further cultivated in vitro and finally incubated with X-Gal as a β-galactosidase substrate that is converted into a insoluble blue coloured product. In microinjection experiments we observe a high frequency of X-Gal stained embryos indicating the occurrence of homologous recombination at the one cell stage or at a later developmental stage. Since these embryos are fixed before the staining procedure it is not possible to further derive mice from them.

B) Generation of Live Mice Carrying the Reporter Gene Cassette

In further experiments, the microinjected zygotes are transferred into pseudopregnant females to allow their further development into live mice (Nagy A, Gertsenstein M, Vintersten K, Behringer R., 2003. Manipulating the Mouse Embryo. Cold Spring Harbour, N.Y.: Cold Spring Harbour Laboratory Press). These experiments show that the microinjected zygotes are able to develop into mouse embryos (FIG. 6) and that the integrated reporter gene is expressed. In one such experiment, microinjected zygotes are transferred into a pseudopregnant female mouse and embryos recovered at day 18 of development. The embryos are euthanized, cut into half and one half is stained with X-Gal staining solution as described above. This analysis reveals that one of six embryos is strongly positive for β-Galactosidase reporter gene activity, as indicated by the blue reaction product.

C) Analysing for Successful Genomic Modification

Without wishing to be bound by the following example, it is envisaged in further experiments to extract genomic DNA from embryonic and newborn, juvenile or adult mice. This DNA can then be analysed for the expected homologous recombination event at the Rosa26 locus by Southern blot analysis using a labelled probe located upstream of the 5′ Rosa26 homology arm of the pRosa26 can then be recognised by a band of 11.5 kb while recombined mice can be identified by the presence of an additional band of 3.65 kb.

D) Generation of Live Mice Harbouring a Venus Reporter Gene Cassette

In a further experiment we used the Rosa26 specific Tal nucleases TalRosa1 and TalRosa2 in combination with the gene targeting vector pROSA26.3-3 (SEQ ID NO: 30), that is equal to pRosa26.8, except that it contains a 1.1 kb reporter cassette for expression of the Venus GFP protein (FIG. 11).

Targeting vector pRosa26.3-3 was used as circular DNA, precipitated and resolved in injection buffer (10 mM Tris, 0.1 mM EDTA, pH 7.2). Tal nuclease RNA for injection was prepared from the linearised expression plasmids pCAG-venus-TalRosa1-Fok-EL and pCAG-venus-TalRosa2-Fok-KK by in vitro transcription from the T7 promoter using the mMessage mMachine kit (Ambion) according to the manufacturer's instructions. The mRNA was further modified by the addition of a poly-A tail using the Poly(A) tailing kit and purified with MegaClear columns from Ambion. Finally the mRNA was precipitated and resolved in injection buffer. Aliquots for injection experiments were adjusted to a concentration of 30 ng/μl of pRosa26.3-3 and 15 ng/μl of each Tal nuclease mRNA. To isolate fertilised oocytes for microinjection, males of the C57BL/6 strain were mated to super-ovulated females of the FVB strain. For super-ovulation three-week old FVB females were treated with 2.5 IU pregnant mares serum (PMS) 2 days before mating and with 2.5 IU Human chorionic gonadotropin (hCG) at the day of mating. Fertilised oocytes were isolated from the oviducts of plug positive females and microinjected in M2 medium (Sigma-Aldrich Inc Cat. No. M7167) with the pRosa26.8-2/ZFN mRNA preparation into one pronucleus and the cytoplasm following standard procedures (Nagy A, Gertsenstein M, Vintersten K, Behringer R., 2003. Manipulating the Mouse Embryo. Cold Spring Harbour, N.Y.: Cold Spring Harbour Laboratory Press).

Microinjected zygotes were transferred into pseudopregnant females to allow their further development into live mice. From adult mice derived from microinjected zygotes genomic tail DNA was extracted for Southern blot analysis. For Southern blot analysis 6 μg of genomic DNA were digested overnight with 30 units BamHI restriction enzyme in a volume of 30 μl and then redigested with 10 units enzyme for 2-3 hours. Samples were loaded on 0.8% agarose gels in TBE buffer and run at 55 V overnight. The gels were then denaturated for one hour in 1.5 M NaCl; 0.5 M NaOH, neutralized for one hour in 0.1 M Tris HCl pH 7.5; 0.5 M NaCl, washed with 2×SSC and blotted overnight with 20×SSC on Hybond N⁺ membranes (GE Healthcare). The membranes were then washed with 2×SSC, UV-crosslinked and stored at −20° C. For hybridization the membranes were preincubated in Church buffer (1% BSA, 1 mM EDTA, 0.5 M phosphate buffer, 7% SDS) for 1 hour at 65° C. under rotation. The Rosa26 5′-probe (SEQ ID NO: 31) was isolated as 460 bp EcoRI fragment from plasmid pCRII-Rosa5′-probe, as described (Hitz, C. Wurst, W., Kuhn, R. 2007. Nucleic Acids Res. 35, e90). As Venus probe the venus coding region, isolated as 730 bp BamHI/EcoRI fragment (SEQ ID NO: 32) from pCS2-venus, was used. DNA fragments used as hybridization probes were heat denatured and labeled with P³² marked dCTP (Perkin Elmer) using the high-prime DNA labeling kit (Roche). Labeled probe DNA was purified on MicroSpin™ S-200 HR columns (GE Healthcare), heat denatured, added to the hybridization buffer and membranes rotated overnight at 65° C. The washing buffer (2×SSC, 0.5% SDS) was prewarmed to 65° C. and the membranes were washed three times (five minutes, 30 minutes, 15 minutes) a 65° C. under shaking. Next, the membranes were exposed at −80° C. to Biomax MS1 films and enhancing sreens (Kodak) for 1-5 days until development. Photos of autoradiographs were taken with a digital camera (Canon) on a transmitting light table and segments excised with the Adobe Photoshop software.

The BamHI digested tail DNA samples were analysed for homologous recombination events at the Rosa26 locus by Southern blot analysis using a labelled probe located upstream of the 5′ Rosa26 homology arm of the pRosa26.3-3 vector. The Rosa26 wildtype allele can then be recognised by a band of 5.8 kb while recombined mice can be identified by the presence of an additional band of 3.1 kb. Using the venus probe and BamHI digestion a 3.9 kb band is detectable (FIG. 11).

In one such experiment tail DNA from 36 pups derived from zygote coinjections of pRosa26.3-3 and TalRosa mRNA revealed the presence of nine recombined Rosa26 alleles, indicated by the presence of an additional, subequimolar band besides the 5.8 kb wildtype Rosa26 fragment (FIG. 12). These recombined Rosa26 alleles appear to be present only in a fraction of cells and exhibit a size of -3.9 kb instead of the predicted size of 3.1 kb. However, due to the use of the Rosa26 5′-probe, that is external to the targeting vector's homology regions, the presence of these bands indicates true recombination activity at Rosa26. All of the recombined tail samples proved positive for the presence of the venus reporter gene, as indicated by the presence of the predicted 3.9 kb BamHI band, detected by the venus hybridization probe (FIG. 12).

We conclude that our Tal nucleases are active in fertilised oocytes and facilitate homologous recombination of a targeting vector with an endogenous locus.

### EXAMPLE 3

Material and Methods

Plasmid Constructions

The gene targeting vector pRosa26.8-2 (SEQ ID NO: 6) was derived from the vector pRosa26.8 bp the removal of a 1.6 kb fragment that contains a pgk-diphtheria toxin A gene. For this purpose pRosa26.8 was digested with EcoRI and KpnI, the vector ends were blunted by treatment with Klenow and T4 DNA polymerase, and the 12.4 kb vector fragment was re-ligated. pRosa26.8 was derived from pRosa26.1 (Soriano P.; Nat Genet 1999; 21:70-71) by insertion of a I-SceI recognition site into the SaclI site located upstream of the 5′ Rosa26 homology arm and the insertion of a splice acceptor element linked to the coding region for β-galactosidase and a polyadenylation signal downstream of the 5′ homology arm. The expression vectors for Tal-finger nucleases recognising a target site within the first intron of the murine Rosa26 locus (SEQ ID NO: 1) are described in example 1 above.

Preparation of DNA and RNA for Microinjection

Plasmid pRosa26.8-2 is linearised by digestion with I-SceI, precipitated and resolved in injection buffer (10 mM Tris, 0.1 mM EDTA, pH 7.2). Tal effector DNA-binding domain nuclease RNA for injection is prepared from the linearised expression plasmids and transcribed from the T7 promoter using the mMessage mMachine kit (Ambion) according to the manufacturers instructions. The mRNA is further modified by the addition of a poly-A tail using the Poly(A) tailing kit and purified with MegaClear columns from Ambion. Finally the mRNA is precipitated and resolved in injection buffer. Aliquots for injection experiments are adjusted to a concentration of 5 ng/μl of pRosa26.8-2 and 2.5 ng/μl of each Tal effector DNA-binding domain—nuclease fusion protein mRNA.

Isolation and Injection of Fertilised Oocytes

To isolate fertilised oocytes, males of the C57BL/6 strain are mated to super-ovulated females of the FVB strain. For super-ovulation three-week old FVB females are treated with 2.5 IU pregnant mares serum (PMS) 2 days before mating and with 2.5 IU Human chorionic gonadotropin (hCG) at the day of mating. Fertilised oocytes are isolated from the oviducts of plug positive females and microinjected in M2 medium (Sigma-Aldrich Inc Cat. No. M7167) with the pRosa26.8-2/Venus-TalRosa1/2-Fok-KK/EL mRNA preparation into one pronucleus and the cytoplasm following standard procedures (Nagy A, Gertsenstein M, Vintersten K, Behringer R., 2003. Manipulating the Mouse Embryo. Cold Spring Harbour, N.Y.: Cold Spring Harbour Laboratory Press).

In Vitro Culture and X-Gal Staining of Embryos

For the detection of β-galactosidase activity the microinjected oocytes are further cultivated for 3 days in KSOM medium (Millipore, Cat. No. MR-020-PD) at 37° C./5% CO₂/5% O₂ and fixed for 10 minutes in 4% formaldehyde in phosphate buffered saline (PBS). After washing with PBS the embryos were transferred to X-Gal staining solution (5 mM K3(Fe^{III}(CN)₆), 5 mM K4(Fe^{II}(CN)₆), 2 mM MgCl₂, 1 mg/ml X-Gal (5-bromo-chloro-3-indoyl-β-D-galactopyranosid) in PBS) and incubated at 37° C. for up to 24 hours.

### EXAMPLE 4

### Construction of Expression and Reporter Vvectors for Tal Nucleases and Determination of Specific Nuclease Activity in Human 293 Cells

Construction of Tal Nuclease Expression Vectors

For the expression of Tal nucleases in mammalian cells we designed the generic expression vector pCAG-Tal-IX-Fok (Seq ID NO: 8) (FIG. 7), that contains a CAG hybrid promoter region and a transcriptional unit comprising a sequence coding for the N-terminal amino acids 1-176 (Seq ID NO: 9) of Tal nucleases, located upstream of a pair of BsmBI restriction sites. This N-terminal region includes an ATG start codon, a nuclear localisation sequence, a FLAG Tag sequence, a glycine rich linker sequence, a segment coding for 110 amino acids of the Tal protein AvrBs3 and the invariable N-terminal Tal repeat of the Hax3 Tal effector. Downstream of the central BsmBI sites, the transcriptional unit contains 78 codons (Seq ID NO: 10) including an invariable C-terminal Tal repeat (34 amino acids) and 44 residues derived from the Tal protein AvrBs3, followed by the coding sequence of the FokI nuclease domain (Seq ID NO: 11) and a polyadenylation signal sequence (bpA). DNA segments coding for arrays of Tal repeats, designed to bind a Tal nuclease target sequence can be inserted into the BsmBI sites of pCAG-Tal-IX-Fok in frame with the up- and downstream coding regions to enable the expression of predesigned Tal-Fok nuclease proteins.

To generate Tal nuclease vectors for expression in mammalian cells we inserted four synthetic DNA segments with the coding regions of four different arrays of Tal repeats (FIG. 7 A-D) into the BsmBI sites of pCAG-Tal-IX-Fok. The four expression vectors pCAG-ArtTal1-Fok (Seq ID NO: 12), pCAG-AvrBs-Fok (Seq ID NO: 13), TalRab1-Fok (Seq ID NO: 14), and TalRab2-Fok (Seq ID NO: 15) enable to express the Tal nucleases ArtTal1-Fok (Seq ID NO: 16), AvrBs-Fok (Seq ID NO: 17), TalRab1-Fok (Seq ID NO: 18), and TalRab2-Fok (Seq ID NO: 19). The Tal element array ArtTal1 recognises the artificial DNA target sequence #1 (FIG. 7A), the Tal array AvrBs recognises the target sequence #2 of the natural AvrBs3 Tal protein (FIG. 7B), whereas the Tal arrays TalRab1 (FIG. 7B) and TalRab2 (FIG. 7B) bind to target sequences #3 and #4 that are derived from the mouse Rab38 gene. The four target sequences were selected such that the binding regions of the Tal nuclease proteins are preceeded by a T nucleotide. Following the sequence downstream of the initial T in the 5′>3′ direction, specific Tal DNA-binding domains were combined together into arrays of 12 (ArtTal1), 17 (AvrBs), 13 (TalRab1) or 14 (TalRab2) Tal elements (FIG. 7).

Construction of Tal Nuclease Reporter Plasmids

To determine the activity and specificity of the four Tal nucleases in mammalian cells we constructed four Tal nuclease reporter plasmids that each contain two copies of one of the four target sequences in inverse orientation, separated by a 15 nucleotide spacer region (FIG. 8a-d). This configuration enables to measure the activity of a single type of Tal nuclease that interacts as a homodimer of two protein molecules that are bound to the inverse pair of target sequences of the reporter plasmid. Upon DNA binding and interaction of the FokI nuclease domains the reporter plasmid DNA double-strand is cleaved within the 15 bp spacer region and exhibits a double-strand break.

The Tal nuclease reporter plasmids contain a CMV promoter region, a 400 bp sequence coding for the N-terminal segment of β-galactosidase and a stop codon. This unit is followed by the Tal nuclease target region (consisting of two inverse oriented recognition sequences separated by a 15 bp spacer region) for ArtTal1-Fok (FIG. 8a), AvrBs-Fok (FIG. 8b), TalRab1-Fok (FIG. 8c), or TalRab2-Fok (FIG. 8d). Within the reporter plasmids ArtTal1-Fok- (Seq ID NO: 20), AvrBs-Fok- (Seq ID NO: 21), TalRab1-Fok- (Seq ID NO: 22), and TalRab2-Fok-Reporter (Seq ID NO: 23), the Tal nuclease target regions are followed by the complete coding region for β-galactosidase and a polyadenylation signal (pA). To test for nuclease activity against the specific target sequence a Tal nuclease expression vector (FIG. 7) was transiently cotransfected with its corresponding reporter plasmid into mammalian cells. Upon expression of the Tal nuclease protein the reporter plasmid is opened by a nuclease-induced double-strand break within the Tal nuclease target sequence (FIG. 8A). The DNA regions adjacent to the double-strand break are identical over 400 bp and can be aligned and recombined by homologous recombination DNA repair (FIG. 8B). Homologous recombination of an opened reporter plasmid will subsequently result into a functional β-galactosidase coding region transcribed from the CMV promoter that leads to the production of β-galactosidase protein (FIG. 8C). In lysates of transfected cells the enzymatic activity of β-galactosidase can be determined by chemiluminescense.

Measurement of Tal Nuclease Activity and Specificity in Human 293 Cells

To determine the activity and specificity of Tal nucleases in mammalian cells, we electroporated one million HEK 293 cells (ATCC #CRL-1573) (Graham F L, Smiley J, Russell W C, Nairn R., J. Gen. Virol. 36, 59-74, 1977) with 5 μg plasmid DNA of one of the Tal nuclease expression vectors (FIG. 7) together with 5 μg of one of the Tal nuclease reporter plasmids (FIG. 8). In addition, each sample received 5 μg of the firefly Luciferase expression plasmid pCMV-hLuc and was adjusted to a total DNA amount of 20 μg with pBluescript (pBS) plasmid DNA. Upon transfection the cells were seeded in triplicate wells of a 6-well tissue culture plate and cultured for two days before analysis was started. For analysis the transfected cells of each well were lysed and the β-galactosidase and luciferase enzyme activities of the lysates were individually determined using chemiluminescent reporter assays following the manufacturer's instruction (Roche Applied Science, Germany) in a luminometer (Berthold Centro LB 960). As positive control we transfected 5 μg of the β-galactosidase expression plasmid pCMVβ with 15 μg pBS, as negative control 5 μg pCMV-hLuc were transfected with 15 μg pBS or 5 μg pCMV-hLuc together with 5 μg of a Tal nuclease reporter plamid and 10 μg pBS. The triplicate β-galactosidase values of each sample were normalised in relation to the levels of Luciferase activity and the mean value and standard deviation of β-galactosidase activity were calculated and expressed in comparison to the pCMVβ positive control defined as 1.0 (FIG. 9). In this type of recombination assay the level of the β-galactosidase catalysed light emission reflects the cleavage and repair of the reporter plasmids and thereby indicates the activity of Tal nucleases.

As shown in FIG. 9A transfection of the pCMV-hLuc and the ArtTal1-Fok- or AvrBs-Fok-Reporter plasmids resulted in very low background levels of β-galactosidase. In contrast, the cotransfection of pCAG-ArtTal1-Fok with the ArtTal1-Fok-Reporter plasmid and the cotransfection of pCAG-AvrBs-Fok with the AvrBs-Fok-Reporter plasmid resulted in a 30-50-fold increase of β-galactosidase activity, indicating the nuclease activity of the Tal nucleases ArtTal1-Fok and AvrBs-Fok. Furthermore, as shown in FIG. 9B, the transfection of the TalRab1-Fok or TalRab2-Fok reporter plasmids without nuclease expression vectors results in a low background level of β-galactosidase, comparable to the transfection of the Luciferase plasmid alone. In contrast, the cotransfection of pCAG-TalRab1-Fok with TalRab1-Fok-Reporter plasmid and of pCAG-TalRab2-Fok with TalRab2-Fok-Reporter plasmid resulted in a strong increase of β-galactosidase activity, indicating the nuclease activity of the Tal nucleases TalRab1-Fok and TalRab2-Fok.

Taken together, these results indicate that the four Tal nucleases develop a strong nuclease activity upon expression in mammalian cells.

To determine whether the observed Tal nuclease activity exhibits specificity for the corresponding nuclease target sequence, we tested the activity of the TalRab1-Fok and TalRab2-Fok nucleases against their authentic target sequence in comparison to an unrelated target sequence. For this purpose the TalRab1-Fok-Reporter plasmid was transfected alone (with pBS), cotransfected with the corresponding expression vector for TalRab1-Fok, or together with the expression vectors for TalRab2-Fok, ArtTal1-Fok or AvrBs-Fok. As shown in FIG. 10, strong nuclease activity developed only in the specific combination of the ArtTal1-Fok expression vector together with the ArtTAl1-Fok reporter plasmid. Vice versa the TalRab1-Fok expression vector did not exhibit nuclease activity against the TalRab2-Fok reporter plasmid.

Taken together, these results indicate that our Tal nucleases are highly specific for the intended target sequences and do not cleave unrelated DNA sequences.

### REFERENCES

Bloch, K. D. (2001). “Mapping by multiple endonuclease digestions.” Curr Protoc Mol Biol Chapter 3: Unit 32.
Boch, J., H. Scholze, et al. (2009). “Breaking the code of DNA binding specificity of TAL-type III effectors.” Science 326(5959): 1509-12.
Bonas, U., R. E. Stall, et al. (1989). “Genetic and structural characterization of the avirulence gene avrBs3 from Xanthomonas campestris pv. vesicatoria.” Mol Gen Genet 218(1): 127-36.
Bradley, A., M. Evans, et al. (1984). “Formation of germ-line chimaeras from embryo-derived teratocarcinoma cell lines.” Nature 309(5965): 255-6.
Brinster, R. L., R. E. Braun, et al. (1989). “Targeted correction of a major histocompatibility class II E alpha gene by DNA microinjected into mouse eggs.” Proc Natl Acad Sci USA 86(18): 7087-91.
Capecchi, M. R. (1989). “The new mouse genetics: altering the genome by gene targeting.” Trends Genet 5(3): 70-6.
Capecchi, M. R. (2005). “Gene targeting in mice: functional analysis of the mammalian genome for the twenty-first century.” Nat Rev Genet 6(6): 507-12.
Cheah, S. S. and R. R. Behringer (2000). “Gene-targeting strategies.” Methods Mol Biol 136: 455-63.
Collins, F. S., J. Rossant, et al. (2007). “A mouse for all reasons.” Cell 128(1): 9-13.
DeChiara, T. M. (2001). “Gene targeting in ES cells.” Methods Mol Biol 158: 19-45.
Doyon, Y., J. M. McCammon, et al. (2008). “Heritable targeted gene disruption in zebrafish using designed zinc-finger nucleases.” Nat Biotechnol 26(6): 702-8.
Durai, S., M. Mani, et al. (2005). “Zinc finger nucleases: custom-designed molecular scissors for genome engineering of plant and mammalian cells.” Nucleic Acids Res 33(18): 5978-90.
Evans, M. J. and M. H. Kaufman (1981). “Establishment in culture of pluripotential cells from mouse embryos.” Nature 292(5819): 154-6.
Geurts, A. M., G. J. Cost, et al. (2009). “Knockout rats via embryo microinjection of zinc-finger nucleases.” Science 325(5939): 433.
Gong, M. and Y. S. Rong (2003). “Targeting multi-cellular organisms.” Curr Opin Genet Dev 13(2): 215-20.
Gu, H., J. D. Marth, et al. (1994). “Deletion of a DNA polymerase beta gene segment in T cells using cell type-specific gene targeting.” Science 265(5168): 103-6.
Hasty, P., A. Abuin, et al. (2000). Gene targeting, principles, and practice in mammalian cells. Gene Targeting: a practical approach. A. L. Joyner. Oxford, Oxford University Press: 1-35.
Hockemeyer, D., F. Soldner, et al. (2009). “Efficient targeting of expressed and silent genes in human ESCs and iPSCs using zinc-finger nucleases.” Nat Biotechnol 27(9): 851-7.
Ivarie, R. (2006). “Competitive bioreactor hens on the horizon.” Trends Biotechnol 24(3): 99-101.
Kamihira, M., K. Nishijima, et al. (2004). “Transgenic birds for the production of recombinant proteins.” Adv Biochem Eng Biotechnol 91: 171-89.
Kandavelou, K. and S. Chandrasegaran (2009). “Custom-designed molecular scissors for site-specific manipulation of the plant and mammalian genomes.” Methods Mol Biol 544: 617-36.
Kay, S., J. Boch, et al. (2005). “Characterization of AvrBs3-like effectors from a Brassicaceae pathogen reveals virulence and avirulence activities and a protein with a novel repeat architecture.” Mol Plant Microbe Interact 18(8): 838-48.
Kay, S. and U. Bonas (2009). “How Xanthomonas type III effectors manipulate the host plant.” Curr Opin Microbiol 12(1): 37-43.
Lai, L. and R. S. Prather (2003). “Creating genetically modified pigs by using nuclear transfer.” Reprod Biol Endocrinol 1: 82.
Maeder, M. L., S. Thibodeau-Beganny, et al. (2008). “Rapid “open-source” engineering of customized zinc-finger nucleases for highly efficient gene modification.” Mol Cell 31(2): 294-301.
Maeder, M. L., S. Thibodeau-Beganny, et al. (2009). “Oligomerized pool engineering (OPEN): an ‘open-source’ protocol for making customized zinc-finger arrays.” Nat Protoc 4(10): 1471-501.
Miller, J. C., M. C. Holmes, et al. (2007). “An improved zinc-finger nuclease architecture for highly specific genome editing.” Nat Biotechnol 25(7): 778-85.
Nagy, A., M. Gertsenstein, et al. (2003). Manipulating the Mouse Embryo. Cold Spring Harbour, N.Y., Cold Spring Harbour Laboratory Press.
Nothias, J. Y., S. Majumder, et al. (1995). “Regulation of gene expression at the beginning of mammalian development.” J Biol Chem 270(38): 22077-80.
Nothias, J. Y., M. Miranda, et al. (1996). “Uncoupling of transcription and translation during zygotic gene activation in the mouse.” EMBO J 15(20): 5715-25.
Palmiter, R. D. and R. L. Brinster (1986). “Germ-line transformation of mice.” Annu Rev Genet 20: 465-99.
Paques, F. and J. E. Haber (1999). “Multiple pathways of recombination induced by double-strand breaks in Saccharomyces cerevisiae.” Microbiol Mol Biol Rev 63(2): 349-404.
Pâques and Duchateau (2007). Meganucleases and DNA double-strand break-induced recombination: perspectives for gene therapy. Curr Gene Ther 7(1): 49-66.
Peippo, J., S. Viitala, et al. (2007). “Birth of correctly genotyped calves after multiplex marker detection from bovine embryo microblade biopsies.” Mol Reprod Dev 74(11): 1373-8.
Porteus, M. H. and D. Baltimore (2003). “Chimeric nucleases stimulate gene targeting in human cells.” Science 300(5620): 763.
Porteus, M. H. and D. Carroll (2005). “Gene targeting using zinc finger nucleases.” Nat Biotechnol 23(8): 967-73.
Rouet, P., F. Smih, et al. (1994). “Expression of a site-specific endonuclease stimulates homologous recombination in mammalian cells.” Proc Natl Acad Sci USA 91(13): 6064-8.
Rouet, P., F. Smih, et al. (1994). “Introduction of double-strand breaks into the genome of mouse cells by expression of a rare-cutting endonuclease.” Mol Cell Biol 14(12): 8096-106.
Roy, A., A. Kucukural, et al. (2010) “I-TASSER: a unified platform for automated protein structure and function prediction.” Nat Protoc 5(4): 725-38.
Santiago, Y., E. Chan, et al. (2008). “Targeted gene knockout in mammalian cells by using engineered zinc-finger nucleases.” Proc Natl Acad Sci USA 105(15): 5809-14.
Schwartzberg, P. L., S. P. Goff, et al. (1989). “Germ-line transmission of a c-abl mutation produced by targeted gene disruption in ES cells.” Science 246(4931): 799-803.
Seibler, J., B. Zevnik, et al. (2003). “Rapid generation of inducible mouse mutants.” Nucleic Acids Res 31(4): e12.
Soriano, P. (1999). “Generalized lacZ expression with the ROSA26 Cre reporter strain.” Nat Genet 21(1): 70-1.
te Riele, H., E. R. Maandag, et al. (1992). “Highly efficient gene targeting in embryonic stem cells through homologous recombination with isogenic DNA constructs.” Proc Natl Acad Sci USA 89(11): 5128-32.
Thomas, K. R. and M. R. Capecchi (1987). “Site-directed mutagenesis by gene targeting in mouse embryo-derived stem cells.” Cell 51(3): 503-12.
Torres, R. M. and R. Kühn (1997). Laboratory protocols for conditional gene targeting. Oxford, Oxford University Press.
Urnov, F. D., J. C. Miller, et al. (2005). “Highly efficient endogenous human gene correction using designed zinc-finger nucleases.” Nature 435(7042): 646-51.
Zambrowicz, B. P., A. Imamoto, et al. (1997). “Disruption of overlapping transcripts in the ROSA beta geo 26 gene trap strain leads to widespread expression of beta-galactosidase in mouse embryos and hematopoietic cells.” Proc Natl Acad Sci USA 94(8): 3789-94.

1. A method of modifying a target sequence in the genome of a eukaryotic cell, the method comprising the step:
(a) introducing into the cell a fusion protein comprising a DNA-binding domain of a Tal effector protein and a non-specific cleavage domain of a restriction nuclease, wherein the restriction nuclease is FokI, or a nucleic acid molecule encoding the fusion protein in expressible form, wherein the fusion protein specifically binds within the target sequence and introduces a double strand break within the target sequence.

2. The method of claim 1, wherein the modification of the target sequence is by homologous recombination with a donor nucleic acid sequence further comprising the step:
(b) introducing a nucleic acid molecule into the cell, wherein the nucleic acid molecule comprises the donor nucleic acid sequence and regions homologous to the target sequence.

3. The method of claim 1 or 2, wherein the cell is selected from the group consisting of a mammalian or vertebrate cell, a plant cell or a fungal cell.

4. The method of any one of claims 1 to 3, wherein the cell is an oocyte.

5. The method of any one of claims 1 to 4, wherein the fusion protein or the nucleic acid molecule encoding the fusion protein is introduced into the cell by microinjection.

6. The method of any one of claims 2 to 4, wherein the nucleic acid molecule of (b) is introduced into the cell by microinjection.

7. The method of any one of claims 1 to 6, wherein the nucleic acid molecule encoding the fusion protein in expressible form is mRNA.

8. The method of any one of claims 2 to 7, wherein the regions homologous to the target sequence are localised at the 5′ and 3′ end of the donor nucleic acid sequence.

9. The method of any one of claims 2 to 8, wherein the regions homologous to the target sequence comprised in the nucleic acid molecule of (b) have a length of at least 400 bp.

10. The method of any one of claims 1 to 9, wherein the modification of the target sequence is selected from the group consisting of substitution, insertion and deletion of a least one nucleotide of the target sequence.

11. The method of any one of claims 1 to 10, wherein the cell is from a mammal selected from the group consisting of rodents, dogs, felides, primates, rabbits, pigs, or cows or wherein the cell is from an avian selected from the group consisting of chickens, turkeys, pheasants, ducks, geese, quails and ratites including ostriches, emus and cassowaries or wherein the cell is from zebrafish.

12. A method of producing a non-human vertebrate or mammal carrying a modified target sequence in its genome, the method comprising transferring a cell produced by the method of any one of claims 1 to 11 into a pseudo pregnant female host.

13. The method of claim 12, further comprising culturing the cell to form a pre-implantation embryo or introducing the cell into a blastocyst prior to transferring it into the pseudopregnant female host.

14. The method of claim 12 or 13, wherein the non-human mammal is selected from the group consisting of rodents, dogs, felides, primates, rabbits, pigs and cows or wherein the vertebrate is selected from the group consisting of fish and avians.

15. A fusion protein comprising a DNA-binding domain of a Tal effector protein and a non-specific cleavage domain of a restriction nuclease, wherein the restriction nuclease is FokI.
